(19) United States
(12) Patent Application Publication
(10) Pub. No.: US 2015/0141389 A1
(43) Pub. Date: May 21, 2015

(54) Topical Formulation Compositions Containing Silicone Based Excipients To Deliver Actives To A Substrate

(71) Applicant: Dow Corning Corporation, Midland, MI (US)

(72) Inventors: Hyder Aliyar, Midland, MI (US); Robert Huber, Midland, MI (US); Gary Loubert, Saginaw, MI (US); Gerald Schalau, Freeland, MI (US)

(21) Appl. No.: 14/391,713

(22) PCT Filed: Mar. 11, 2013
(86) PCT No.: PCT/US13/30212

§ 371 Date: Oct. 10, 2014

Related U.S. Application Data
(60) Provisional application No. 61/639,611, filed on Apr. 27, 2012.

(51) Int. Cl. A61K 47/34; A61K 31/192; A61K 47/06; A61K 31/573; A61K 47/10; A61K 47/12; A61K 47/44; A61K 31/196; A61K 9/00; A61K 47/24

(52) U.S. Cl. CPC A61K 47/34; A61K 9/0014; A61K 31/192; A61K 47/06; A61K 47/24; A61K 47/10; A61K 47/12; A61K 47/44; A61K 31/196; A61K 31/573
USPC 514/180; 514/570; 514/567; 514/179

(57) ABSTRACT

The present disclosure relates to a semi-solid topical drug delivery formulation including a silicone-based excipient, at least one volatile solvent, at least one active configured to be topically delivered through a patient's skin for an intended therapeutic application, and at least one enhancer. The formulation may additionally optionally include at least one agent that provides occlusivity when the formulation is applied onto a patient's skin. The at least one active may be a healthcare and/or pharmaceutical active.

### FIELD OF THE INVENTION

The present disclosure relates to topical formulation compositions containing silicone-based excipients to deliver pharmaceutical, personal care or healthcare actives to a substrate, such as mammalian skin.

### BACKGROUND OF THE INVENTION

Traditionally, most active ingredients and pharmaceuticals have been delivered to patients via oral ingestion or injection. Active ingredients or drugs delivered via oral ingestion may take a certain amount of time before they start delivering a therapeutic effect or they may deliver a therapeutic effect for only a short amount of time. Additionally, some people have difficulty ingesting a drug, especially if the drug is included in a relatively large sized pill. Another reason that some oral drug delivery may be problematic is due to high first-pass metabolism. There are a variety of problems associated with injections as well. Most importantly, a majority of people do not enjoy receiving injections. Topically applied formulations avoid a variety of concerns associated with oral and intravenous application methods, including avoidance of first-pass metabolism, possible gastro-intestinal incompatibility and varied conditions of absorption, like pH changes, presence of enzymes, and gastric emptying times. Moreover, topically applied formulations may provide several additional advantages including lower fluctuations in plasma drug levels, ability to more selectively target a specific site for treatment, and ease of treatment. For some conditions, the most effective way to deliver an active is by applying such active directly to the source. Topical formulations that previously described in the prior art still possess several significant limitations such as poor permeability of the drug through the skin from the formulation, low efficiency in delivering the drug by the formulation, residual drugs in the formulation post-application, poor wear characteristics that decrease delivery efficacy and patience compliance, and poor aesthetics that also lead to poor patience compliance. Therefore, there is a need for a new class of topical formulations that improve and overcome the limitations discussed above.

While some topical formulations have been developed in the art to deliver actives to the skin, such formulations have suffered from several important shortcomings. Most significantly, such formulations have been unable to deliver a therapeutic amount of the active ingredient to the skin for an extended period of time. Such formulations tend to deliver a therapeutic amount of the pharmaceutical or healthcare active only for a short period of time—such as for about one or two hours—and the amount of active that is delivered to the skin after one or two hours drops off dramatically, such that little or no therapeutic effect is achieved after about two hours following application to the substrate. Another shortcoming of many formulations known in the art is that they contain water, which requires the use of a significant number of preservatives to prevent or inhibit bacterial growth. Preservatives may be undesirable to some people or in certain applications.

Therefore, there is currently a significant need for a topical formulation that can deliver a therapeutic amount of an active ingredient to the skin for an extended period of time, such as for more than about four, eight or up to 24 hours. Additionally, there is currently a need for a topical formulation that is capable of being free or substantially free of preservatives. Moreover, the active ingredient has to be uniformly incorporated into the topical formulation; in other words, the active ingredient should not include any agglomerates. Finally, the topical formulation should maintain an aesthetic profile and pleasant sensory upon application.

### SUMMARY OF THE INVENTION

A controlled release semi-solid topical drug delivery formulation is disclosed. The controlled-release formulation is for topical application of an active ingredient to a substrate, such as mammalian skin. The topical formulation provides increased penetration (flux) into the skin of the active ingredient dissolved or dispersed in the formulation compared to the topical formulations currently available in the art.

The formulation prepared according to the present disclosure may include a silicone-based excipient, at least one volatile solvent, at least one active configured to be topically delivered through a patient's skin for an intended therapeutic application, and at least one enhancer. In an alternative embodiment, the formulation may additionally optionally include at least one agent that is configured to provide occlusivity when the formulation is applied onto the patient's skin. The at least one active may be a pharmaceutical, personal care and/or a healthcare active.

The silicone-based excipient may be a silicone elastomer blend, a silicone organic elastomer blend, a silicone resin, a silicone elastomer, a pressure sensitive adhesive, a silicone gum, or any combination thereof. The silicone-based excipient may be a silicone elastomer blend, or a silicone organic elastomer blend included in a silicone or organic carrier fluid such as isododecane, cyclopentasiloxane, isodecylneopentanoate, caprylyl methicone, isopropyl alcohol, propylene glycol, and any combination thereof. According to another aspect, the silicone-based excipient may be a dimethicone cross polymer, a dimethicone/bis-isobutyl propylene glycol cross polymer, a polyethylene glycol-12 dimethicone/bis-isobutyl propylene glycol-20 cross polymer, or any combination thereof.

Advantageously, the topical formulation according to the present disclosure may be anhydrous and may be free or substantially free of preservatives. The topical formulation may be configured to deliver a therapeutic amount of pharmaceutical or healthcare active to the substrate such as skin for an extended period of time. Alternatively, the topical formulation may be configured to deliver a therapeutic amount of pharmaceutical or healthcare active to a substrate, such as mammalian skin, for more than four, or, alternatively, for more than eight hours.

Additional aspects of the disclosure will be apparent to those of ordinary skill in the art in view of the detailed description of various embodiments, a brief description of which is provided below.

### BRIEF DESCRIPTION OF THE DRAWINGS

FIG. 1 is a flux profile for silicone organic elastomer blend based formulation examples 1-3 including Ibuprofen and a commercial benchmark including Ibuprofen.

FIG. 1A is a flux profile for silicone elastomer blend based formulation example 3A and a commercial benchmark including ibuprofen.

FIG. 2 is a flux profile for Petrolatum based formulation examples 4-6 including Ibuprofen and a commercial benchmark including Ibuprofen.

FIG. 3 is a flux profile for Carbopol® 971P NF based formulation examples 7-9 including Ibuprofen and a commercial benchmark including Ibuprofen.

FIG. 4 is a flux profile for Eudragit® E100 based formulation examples 10-12 including Ibuprofen and a commercial benchmark including Ibuprofen.

FIG. 5 is a flux profile for Eudragit® S100 based formulation examples 13-15 including Ibuprofen and a commercial benchmark including Ibuprofen.

FIG. 6 is a flux profile for Eudragit® L100 based formulation examples 16-18 including Ibuprofen and a commercial benchmark including Ibuprofen.

FIG. 7 is a flux profile for Eudragit® L100-55 based formulation examples 19-21 including Ibuprofen and a commercial benchmark including Ibuprofen.

FIG. 8 is a flux profile for silicone organic elastomer blend based formulation examples 22-26 including diclofenac sodium and a commercial benchmark including diclofenac sodium.

FIG. 9 is a flux profile for silicone elastomer blend based formulation examples 27 and 28 including diclofenac sodium and a commercial benchmark including diclofenac sodium.

FIG. 10 is a flux profile for silicone organic elastomer blend based formulation example 29, silicone elastomer based formulation example 30, carbopol based formulation 31, all including clobetasol propionate and a commercial benchmark including clobetasol propionate.

FIG. 11 is a cumulative release profile for silicone gum based formulation examples 32-34 including ibuprofen, silicone elastomer blend based formulation examples 2 and 3A including ibuprofen, and a commercial benchmark including ibuprofen.

FIG. 12 is a cumulative release profile for silicone gum based formulation examples 35-37 including hydrocortisone, silicone elastomer blend based formulation examples 38 and 39 including hydrocortisone, and a commercial benchmark including hydrocortisone.

FIG. 13 is a cumulative release profile for silicone elastomer blend based formulation examples 40-42 and a commercial benchmark including ibuprofen.

### DETAILED DESCRIPTION

Features and advantages of the present disclosure will now be described with occasional reference to specific embodiments. However, the invention may be embodied in different forms and should not be construed as limited to the embodiments set forth herein. Rather, these embodiments are provided so that this disclosure will be thorough and complete and will fully convey the scope of the disclosure to those skilled in the art.

Unless otherwise indicated or defined, all technical and scientific terms used herein have the same meaning as commonly understood by one of ordinary skill in the art to which the invention pertains. The terminology used herein is for describing particular embodiments only and is not intended to be limiting. The term “ambient conditions” as used throughout the specification refers to surrounding conditions under about one atmosphere of pressure, at about 50% relative humidity, and at about 25° C., unless otherwise specified.

Unless otherwise indicated, all numbers expressing quantities of ingredients, properties such as molecular weight, % by weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of this disclosure. Notwithstanding that the numerical rangers and parameters setting forth the broad scope of the disclosure are approximations, the numerical values set forth in the specific examples are reported as precisely as possible. Any numerical values, however, inherently contain errors necessarily resulting from error found in their respective measurements.

All percentages, parts, and ratios are based upon the total weight of the topical formulation, unless otherwise specified. All such weights as they pertain to listed ingredients are based on the active level and, therefore, do not include carriers or by-products that may be included in commercially available materials, unless otherwise specified.

The substrate is typically a biological surface, human body tissue, and/or animal body tissue. More specific substrates include, but are not limited to, skin, hair, mucous membrane, teeth, nails, and eyes.

The formulation prepared according to the present disclosure is typically applied for topical therapy, such as to treat damaged or diseased skin, and wound care, such as to treat cuts, burns, scars, and the like, with a dressing formed from, or including, the controlled-release topical formulation where the silicone-based excipient functions as a substantive cream or a liquid bandage that continuously delivers the active agent to the substrate. The present disclosure, including films formed by the controlled-release formulations of the present disclosure, may also be applied in various transdermal, pharmaceutical, veterinary, and oral health care applications. It may be used as an in situ formed patch standing by itself, or it can be protected with a secondary film, dressing, or patch, or it can be part of a more complex construction such as a transdermal patch or wound dressing. As indicated above, the controlled-release formulation, which is hereafter referred to as the composition or the formulation, includes the silicone-based excipient and the active agent. The active agent is uniformly incorporated into or dispersed in the topical formulation. The topical formulations may be spread, sprayed, or otherwise dispersed on to the substrate such as skin or other tissue.

The topical formulation may be prepared by mixing (a) a silicone-based excipient, (b) at least one volatile solvent, (c) at least one pharmaceutical active configured to be topically delivered through a patient's skin for an intended therapeutic application, and (d) at least one enhancer. The topical formulation may also optionally include (e) at least one agent configured to provide occlusivity when the formulation is applied to the patient's skin. The silicone-based excipient may be contained in a suitable carrier fluid.

The formulation according to the present disclosure may include between about 2 and about 80% by weight of the silicone-based excipient. Alternatively, the formulation may include between about 10 and about 50% by weight of the silicone-based excipient.

The formulation according to the present disclosure may include between about 10 and about 80% by weight of the at least one volatile solvent. Alternatively, the formulation may include between about 20 and about 60% by weight of the at least one volatile solvent. The at least one volatile solvent may include one solvent or a mixture of solvents as selected by one of ordinary skill in the art.

The amount of healthcare or pharmaceutical active present in the topical formulation may vary. The formulation may include between about 0.001 to 50% by weight of the active. Alternatively, the formulation may include between about 0.05 to about 25% by weight of the active. Alternatively, the formulation may include between about 0.05 to about 10% by weight of the active.

The formulation according to the present disclosure may include between about 0 and about 80% by weight of the at least one enhancer. Alternatively, the formulation may include between about 0.5 and about 50% by weight of the at least one enhancer.

In one embodiment, the enhancer may include a non-volatile excipient and a skin penetration enhancer and the weight ratio of the non-volatile excipient to the penetration enhancer in the final formulation may be from about 100:1 to about 50:50. Alternatively, the formulation may include between about 0.5 to about 50% by weight of the penetration enhancer. In yet another embodiment, the formulation may include between about 20 to about 40% by weight of the non-volatile excipient.

The formulation according to the present disclosure may additionally include between about 0 and about 50%% by weight of the at least one agent configured to provide occlusivity. Alternatively, the formulation may include between about 0.5 to about 25% by weight of the agent configured to provide occlusivity.

Silicone-Based Excipient

The silicone-based excipient may be any silicone-containing polymer material, including a silicone elastomer blend, a silicone organic elastomer blend, a silicone resin, a silicone elastomer, a pressure sensitive adhesive, a silicone gum, a silicone wax, an elastomer base sealant, adhesive or any combination thereof. The silicone-based excipient may be a dimethicone cross polymer, a dimethicone/bis-isobutyl propylene glycol cross polymer, a polyethylene glycol-12 dimethicone/bis-isobutyl propylene glycol-20 cross polymer, or any combination thereof.

Silicones are a class of compounds based on polydialkylsiloxanes. Silicones have been used extensively to enhance aesthetics of personal care formulations by providing a unique sensory profile upon application. Silicone elastomer gels are generally obtained by a crosslinking hydrosilylation reaction of a SiH polysiloxane with another polysiloxane containing an unsaturated hydrocarbon substituent, such as a vinyl functional polysiloxane, or by crosslinking a SiH polysiloxane with a hydrocarbon diene. The silicone elastomers may be formed in the presence of a carrier fluid, such as a volatile silicone, resulting in a gelled formulation.

The silicone-based excipient may be a pressure sensitive adhesive (PSA). The PSA may be the reaction product of a hydroxyl end-blocked polydimethylsiloxane polymer and a hydroxy functional silicate resin. The polymer and resin react in a condensation reaction to form the PSA. The advantage of using the PSA as the silicone component is the substantivity that the PSA provides. The substantivity is particularly advantageous in human and veterinary applications that require significant substantivity for the active agent to provide sustained pharmacological effects.

For purposes of the present disclosure, the terms “silicone rubber” and “silicone elastomer” are synonymous, at least to the extent that both silicone components are capable of elongation and recovery. The silicone elastomers may be contained in a carrier fluid such as cyclopentasiloxane, isododecane, isodecylneopentanoate, caprylyl methicone, or other suitable carrier fluids. Silicone rubbers and silicone elastomers are generally crosslinked or reacted silicone polymers. In contrast, silicone gums are capable of being stretched, but they do not generally snap back. Silicone gums are the high molecular weight, generally linear, polydiorganosiloxanes that can be converted from their highly viscous plastic state into a predominately elastic state by crosslinking. Silicone gums are often used as one of the main components in the preparation of silicone rubbers and silicone elastomers.

The silicone resins may include MQ resins. The acronym MQ as it relates to silicone resins is derived from the symbols M, D, T, and Q each of which represent a functionality of different types of structural units which may be present in silicone resins containing siloxane units joined by Si—O—Si bonds. Monofunctional (M) unit represents (CH₃)₃SiO_{1/2}. Difunctional (D) unit represents (CH₃)₂SiO_{2/2}. Trifunctional (T) unit represents CH₃SiO_{3/2} and results in the formation of branched linear siloxanes. Tetrafunctional (Q) unit represents SiO_{4/2} which results in the formation of crosslinked and resinous silicone compositions. Hence, MQ is used when the siloxane contains all monofunctional M and tetrafunctional Q units, or at least a high percentage of M and Q units such as to render the silicone resinous.

Silicone resins may include non-linear siloxane resins having a glass transition temperature (Tg) above about 0° C. Glass transition temperature is the temperature at which an amorphous material such as a higher silicone polymer changes from a brittle vitreous state to a plastic state. The silicone resin generally has the formula R′ₐSiO_{(4-a)/2} wherein R′ is a monovalent hydrocarbon group with 1-6 carbon atoms or a functionally substituted hydrocarbon group with 1-6 carbon atoms, and a has an average value of 1-1.8. The silicone resin will preferably include monofunctional (M) units R″₃SiO_{1/2} and tetrafunctional (Q) units SiO_{4/2}, in which R″ is the monovalent hydrocarbon group having 1-6 carbon atoms, most preferably the methyl group. The number ratio of M groups to Q groups may be in the range of 0.5:1 to 1.2:1, so as to provide an equivalent wherein a in the formula R′ₐSiO_{(4-a)/2} has an average value of 1.0-1.63. The number ratio of M groups to Q groups may also be between about 0.6:1 to about 0.9:1. Silicone MQ resins in which the number of Q units per molecule is higher than 1 or higher than 5 may also be used.

The silicone resin may also contain between about 1 to about 5% by weight of silicon-bonded hydroxyl radicals such as a dimethylhydroxysiloxy unit (HO)(CH₃)₂SiO_{1/2}. If desired, the silicone resin may contain minor amounts of difunctional (D) units and/or trifunctional (T) units. Silicone resins having a viscosity of at least 100,000,000 (100 million) centistoke (mmf²/s) and a softening temperature of less than about 200° C. may also be used. The silicone resin may include (i) silicone resins of the type MₓQ_{y} where x and y have values such that the silicone resin contains at least more than 5 Q units per molecule; (ii) silicone resins of the type MₓT_{y} where x and y have values such that the silicone resin contains at least more than 5 T units per molecule; and (iii) silicone resins of the type MₓD_{y}TₚQₚ where x, y, p, and q have values such that the sum of Q and T units is at least more than 5 units per molecule, and the number of D units varies from 0-100.

Volatile Solvent

The formulation according to the present disclosure includes a volatile solvent. The silicone-based excipient may be contained in volatile solvent (or carrier fluid) to provide the present topical formulations. Typically, the volatile solvent is the solvent used for conducting the hydrosilylation reaction to form the silicone-based excipient. Suitable volatile solvents include volatile solvents, organic liquids (oils and solvents), silicones and mixtures thereof.

Solvents may include volatile liquids such as alcohols (e.g., methyl, ethyl, isopropyl alcohols and methylene chloride); ketones (e.g., acetone); aromatic hydrocarbons such as benzene derivatives (e.g., xylenes and toluenes); lower molecular weight alkanes and cycloalkanes (e.g., hexanes, heptanes and cyclohexanes); and alkanoic acid esters (e.g., ethyl acetate, n-propyl acetate, isobutyl acetate, n-butyl acetate, isobutyl isobutyrate, hexyl acetate, 2-ethylhexyl acetate or butyl acetate); and combinations and mixtures thereof.

Typically, the volatile solvent is an organic liquid. Organic liquids include oils and solvents. The organic liquids are exemplified by, but not limited to, aromatic hydrocarbons, aliphatic hydrocarbons, alcohols, aldehydes, ketones, amines, esters, ethers, glycols, glycol ethers, alkyl halides and aromatic halides. Hydrocarbons include, isododecane, isohexadecane, Isopar L (C11-C13), Isopar H (C11-C12), hydrogentated polydecene. Ethers and esters include, isodecyl neopentanoate, neopentylglycol heptanoate, glycol distearate, dicaprylyl carbonate, diethylhexyl carbonate, propylene glycol n butyl ether, ethyl-3 ethoxypropionate, propylene glycol methyl ether acetate, tridecyl neopentanoate, propylene glycol methylether acetate (PGMEA), propylene glycol methylether (PGME). octyldodecyl neopentanoate, diisobutyl adipate, diisopropyl adipate, propylene glycol dicaprylate/dicaprate, and octyl palmitate. Additional volatile solvents suitable as a standalone compound or as an ingredient to the carrier fluid include fats, oils, fatty acids, and fatty alcohols.

The volatile solvent may also be a low viscosity organopolysiloxane or a volatile methyl siloxane or a volatile ethyl siloxane or a volatile methyl ethyl siloxane having a viscosity at 25° C. in the range of about 1 to about 1,000 mm²/sec, exemplified by hexamethylcyclotrisiloxane, octamethyleyelotetrasiloxane, decamethylcyclopentasiloxane, dodecamethylcyclohexasiloxane, octamethyltrisiloxane, decamethyltetrasiloxane, dodecamethylpentasiloxane, tetradecamethylhe xasiloxane, hexadeamethylheptasiloxane, heptamethyl-3-{(trimethylsilyl)oxy)}trisiloxane, hexa methyl-3,3,bis{(trimethlylsilyl)oxy}trisiloxane pentamethyl{(trimethylsilyl)oxy}cyclotrisiloxane as well as polydimethylsiloxanes, polyethylsiloxanes, polymethylethylsiloxanes, polymethylphenylsiloxanes, polydiphenylsiloxanes.

Enhancer

In addition to active agent and silicone-based excipients, various excipients and/or enhancing agents may be incorporated into the topical formulation. As generally understood by those skilled in the art, excipients are additives that are used to convert the active agent into appropriate dosage forms that are suitable for application to the substrate. Excipients may also be added to stabilize the formulation and to optimize application characteristics, such as flowability.

Examples of potential excipients include, but are not limited to, excipients that are found in the Cosmetics, Toiletry, Fragrance Association (CTFA) ingredient Database and the handbook of pharmaceutical excipients such as absorbents, anticaking agents, antioxidants (such as, ascorbic acid, ascorbic acid polypeptide, ascorbyl dipalmitate, BHA, BHT, magnesium ascorbate, magnesium ascorbyl phosphate, propyl gallate sodium ascorbate, sodium ascorbyl/cholesteryl phosphate, sodium bisulfite, sodium erythorbate, sodium metabisulfide, tocopheryl acetate, tocopheryl nicotinate), antistatic agents, astringents, binders, buffering agents, bulking agents, chelating agents, colorants, cosmetic astringents, biocides (such as parabens, organic acids, organic bases, alcohols. isothiazolinones and others), deodorant agents, emollients, external analgesics (such as Benzyl Alcohol, Methyl Salicylate, Camphor, Phenol, Capsaicin, Juniper Tar (Menthol, Resorcinol, Methyl Nicotinate, and Turpentine Oil), film formers, flavoring agents, fragrance ingredients, humectants, lytic agents, moisturizing agents, occlusivity enhancers, opacifying agents, oxidizing agents (such as Peroxides, Bromates, Chlorates, Potassium Iodates, and Persulfates,), reducing agents (such as Sulfites, Thioglycolates, Cystein, Cysteine HCl, Glutathione, Hydroquinone, Mercaptopropionic Acid, Sulfonates, Thioglycolic Acid), penetration enhancers, pesticides, plasticizers, preservatives, skin bleaching agents such as hydroquinone, skin conditioning agents, skin protectants (such as Allantoin, Aluminum Acetate, Dimethicone, Glycerin, Kaolin, Lanolin, Mineral Oil, Petrolatum, Talc, and Zinc Oxide), slip modifiers, solubilizing agents, solvents, sunscreen agents (such as Aminobenzoic Acid, Cinoxate, cinnamates, Aminobenzoates, Oxybenzone, Red Petrolatum, Titanium Dioxide, and Trolamine Salicylate), surface modifiers, surfactants and emulsifying agents, suspending agents, thickening agents, viscosity controlling agents including increasing or decreasing agents, UV light absorbing agents (such as Acetaminosalol, Allatoin PABA, Benzalphthalide, and Benzophenone,). Other possible excipients include, but are not limited to, sugars and derivatives (such as acacia, dextrin, dextrose, maltodextrin, and sorbitol), starch derivatives, cellulosic materials (such as methyl cellulose, Ethylcellulose, Hydroxyethylcellulose, Hydroxypropylcellulose, and Hydroxypropylmethylcellulose,), polysaccharides (such as dextrates, guar gum, and xanthan gum), polyethers, suspending agents cyclodextrins, and others

Enhancers may also be exemplified by monohydric alcohols such as ethanol and isopropyl, butyl and benzyl alcohols, or dihydric alcohols such as ethylene glycol, diethylene glycol, or propylene glycol, dipropylene glycol and trimethylene glycol, or polyhydric alcohols such as butylene glycol, hexylene glycol, polypropylene glycol, ethylene glycol, and polyethylene glycol, which enhance drug solubility; polyethylene glycol ethers of aliphatic alcohols (such as cetyl, lauryl, oleyl and stearyl) including polyoxyethylene (4) lauryl ether, polyoxyethylene (2) oleyl ether and polyoxyethylene (10) oleyl ether commercially available under the trademark BRIJ® 30, 93 and 97, respectively, from Uniqema Americas LLC (Wilmington, Del.), and others such as BRIJ® 35, 52, 56, 58, 72, 76, 78, 92, 96, 700 and 721; vegetable, animal and fish fats and oils such as olive, and castor oils, squalene, lanolin; fatty acids such as oleic, linoleic, and capric acid, and the like; fatty acid esters such as propyl oleate, decyl oleate, isopropyl palmitate, glycol palmitate, glycol laurate, dodecyl myristate, isopropyl myristate and glycol stearate which enhance drug diffusibility; fatty acid alcohols such as oleyl alcohol and its derivatives; fatty acid amides such as oleamide and its derivatives; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide and dimethylformamide which affect keratin permeability; salicylic acid; amino acids; benzyl nicotinate; and higher molecular weight aliphatic surfactants such as lauryl sulfate salts; and esters of sorbitol and sorbitol anhydride such as polysorbate 20 commercially available under the trademark Tween® 20 from Uniqema Americas LLC (Wilmington, Del.), as well as other polysorbates such as 21, 40, 60, 61, 65, 80, 81, and 85. Other enhancers include enzymes, panthenol, and other non-toxic enhancers commonly used in transdermal or transmucosal compositions.

Polyhydric alcohols also include glycols, triols and polyols having 4 to 6 alcoholic hydroxyl groups. Typical of said glycols are glycols containing 2 to 6 carbon atoms, e.g. ethylene glycol, propylene glycol, butylene glycol, polyethylene glycol (average molecular weight about 200-8,000, preferably about 200 to 6,000), etc. Examples of said triols include glycerin, trimethylolpropane, etc. Said polyols are exemplified by sorbitol, polyvinylpyrrolidone, etc. These polyhydric alcohols may be used either singularly or in combination (preferably, of two or three). Thus, for example, glycerin or dipropylene glycol alone, or a mixture of either glycerin or dipropylene glycol with butylene glycol can be employed.

Active Ingredient

The formulation may include an active selected from any personal, healthcare, or pharmaceutical active. As used herein, a “personal care active” means any compound or mixtures of compounds that are known in the art as additives in the personal care formulations that are typically added for treating hair or skin to provide a cosmetic and/or aesthetic benefit. A “healthcare active” means any compound or mixtures of compounds that are known in the art to provide a pharmaceutical or medical benefit. Thus, “healthcare active” includes materials considered as an active ingredient or active drug ingredient as generally used and defined by the United States Department of Health & Human Services Food and Drug Administration, contained in Title 21, Chapter I, of the Code of Federal Regulations, Parts 200-299 and Parts 300-499.

Thus, active ingredient can include any component that is intended to furnish pharmacological activity or other direct effect in the diagnosis, cure, mitigation, treatment, or prevention of disease, or to affect the structure or any function of the body of a human or other animals. The phrase can include those components that may undergo chemical change in the manufacture of drug products and be present in drug products in a modified form intended to furnish the specified activity or effect.

Some representative examples of pharmaceutical or healthcare active ingredients include non-steroidal anti-inflammatory drug, a steroid, a retinoid, an azole, traditional Chinese medicines, anti-acne, antibiotics, or any combination thereof.

The active ingredient can include a water-soluble or an oil-soluble active drug ingredient. Representative examples of some suitable water-soluble active drug ingredients which can be used are hydrocortisone, ketoprofen, morphine, hydromorphone, heparin, penicillin G, 5-fluorouracil, 6-azauridine, 6-thioguanine, niacinamide, salicylic acid, and ketoconazole.

Representative examples of some suitable oil-soluble active drug ingredients are clonidine, scopolamine, nitroglycerin, ibuprofen, indomethacin, naproxen, and steroids.

Active ingredients for purposes of the present invention also include anti-acne agents such as benzoyl peroxide and tretinoin; anti-inflammatory agents; corticosteroidal drugs; non-steroidal anti-inflammatory agents such as diclofenac; anesthetic agents such as lidocaine; antipruritic agents; and antidermatitis agents.

Some additional representative examples of active ingredients include minerals; hormones; topical antimicrobial and antibacterial agents such as chlorohexadiene gluconate agents and antibiotic active ingredients, antifungal active ingredients, such as miconazole nitrate; astringent active ingredients; deodorant active ingredients; wart remover active ingredients; corn and callus remover active ingredients; pediculicide active ingredients for the treatment of head, pubic (crab), and body lice; active ingredients for the control of dandruff, seborrheic dermatitis, or psoriasis, such as clobetasol propionate; and sunburn prevention and treatment agents.

The active agent may include a lipophilic drug and/or hydrophilic drug. Whether or not the active agent is a lipophilic drug or a hydrophilic drug, other possible active agents include, but are not limited to, antiacne agents, such as sulfur, antiseptic, and povidone-iodine, antibacterial, antimicrobial agents, such as alcohol, benzalkonium chloride, benzethonium chloride, phenol, silver ions, nanocrystalline silver, anticancer agents, smoking cessation compositions, histamine blocker, bronchodilator, analgesic, antihistamine, alpha-I blocker, beta blocker, ACE inhibitor, sedative, tranquillizer, anticoagulant agents, vitamins, antiaging agents, anticellulites, cell growth nutrients, perfumes, shaving products, therapeutic active agents such as penicillins, tetracyclines, aspirin, acetominophen, catecholamines, procaine, lidocaine, lidocaine HCL, benzocaine, sulphonamides, ticonazole, and retinol, Drugs affecting renal and cardiovascular function drugs affecting gastrointestinal function, drugs for the treatment of helminthiasis (such as thiabendazole and mebendazole), drugs for the treatment of microbial diseases (such as ciprofloxacin, penicillin G nafcillin, minocycline, clindamycin, acyclovir and ganciclovir), drugs for the treatment of nutrient deficiency (such as folic acid, niacinamide, ascorbic acid and thiamine), drugs for hormonal replacement therapy (such as estradiol, ethinyl estradiol and norethindrone), drugs that inhibit the synthesis and actions of adrenocortical hormones (such as cortisol, cortisone and prednisone), and drugs used in dermatology for the treatment of dermatoses (such as betamethasone dipropionate, hydrocortisone, dexamethasone sodium phosphate, tretinoin, isotretinoin, dapsone, calipotriene, and arotinoid).

Useful active ingredients for use in formulations according to the present disclosure include vitamins and its derivatives, including “pro-vitamins.” Vitamins useful herein include, but are not limited to, Vitamin A₁, retinol, C₂-C₁₈ esters of retinol, vitamin E, tocopherol, esters of vitamin E, and mixtures thereof. Retinol includes trans-retinol, 1,3-cis-retinol, 11-cis-retinol, 9-cis-retinol, and 3,4-didehydro-retinol, Vitamin C and its derivatives, Vitamin B₁, Vitamin B₂, Pro Vitamin B₅, panthenol, Vitamin B₆, Vitamin B₁₂, niacin, folic acid, biotin, and pantothenic acid. Other suitable vitamins and the INCI names for the vitamins considered included herein are ascorbyl dipalmitate, ascorbyl methylsilanol pectinate, ascorbyl palmitate, ascorbyl stearate, ascorbyl glucocide, sodium ascorbyl phosphate, sodium ascorbate, disodium ascorbyl sulfate, potassium (ascorbyl/tocopheryl) phosphate.

The active component of the present invention can be a protein, such as an enzyme. The internal inclusion of enzymes in these formulations has the advantages of preventing enzymes from deactivating and maintaining bioactive effects of enzymes for a longer time period. Enzymes include, but are not limited to, commercially available types, improved types, recombinant types, wild types, variants not found in nature, and mixtures thereof. For example, suitable enzymes include hydrolases, cutinases, oxidases, esterases, lactases, peroxidases, and mixtures thereof. Hydrolases include, but are not limited to, proteases (bacterial, fungal, acid, neutral or alkaline), amylases (alpha or beta), lipases, cellulases, collagenases, lisozyrnes, and mixtures thereof. Said protease include, but are not limited to, trypsin, chymotrypsin, pepsin, pancreatin and other mammalian enzymes; papain, bromelain and other botanical enzymes; subtilisin, epidermin, nisin, naringinase(L-rhammnosidase) urokinase and other bacterial enzymes. Said lipase include, but are not limited to, triacyl-glycerol lipases, monoacyl-glycerol lipases, lipoprotein lipases, e.g. steapsin, erepsin, pepsin, other mammalian, botanical, bacterial lipases and purified ones. Natural papain is included as said enzyme. Further, stimulating hormones, e.g. insulin, can be used together with these enzymes to boost their effectiveness.

The pharmaceutical or healthcare active may also include one or more plant extracts. Examples of these components are as follows: t, Ginkgo Biloba extract, oolong tea extract, Echinacea extract, Scutellaria root extract, Phellodendro bark extract, Watercress extract, Chamomile extract, Horsetail extract, lemon extract, Chinese milk vetch extract, rose extract, rosemary extract, Roman Chamomile extract royal jelly extract or any other botanical extract that may be topically applied to achieve a pharmaceutical outcome.

The active ingredient may be selected depending on the application for which the topical formulation is used. For example, if the desired effect is pain relief, ibuprofen may be used as the active. If the desired effect is acne prevention and control, benzoyl peroxide may be used.

Occlusivity Agent

The formulation may include an occlusivity agent configured to provide occlusivity when the formulation is applied on top of the skin. The occlusivity agent may include petrolatum, organic wax, silicone wax, polyacrylates and methacrylates (exemplified by, but not limited to Eudragit® E100, S100, L100, and L100-55), polyvinyl pyrolidone, polyvinyl alcohol, vinylacetate-vinylpyrolidone copolymer, or any combination thereof. A majority of film-forming polymers can be considered to provide occlusive properties to the formulation and thus any suitable film-forming polymer may be used in the present formulation.

The occlusivity agent may be a wax or a wax-like material. The waxes or wax-like materials useful in the formulation according to the present disclosure generally have a melting point range of about 35 to 120° C. at atmospheric pressure. Waxes in this category include synthetic wax, ceresin, paraffin, ozokerite, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, or mixtures thereof. Additionally, the occlusivity agent may include waxes capable of being used as non-silicone fatty substances, animal waxes, such as beeswax; vegetable waxes, such as carnauba, candelilla wax; mineral waxes, such as paraffin or lignite wax; microcrystalline waxes; ozokerites; synthetic waxes, including polyethylene waxes, and waxes obtained by the Fischer-Tropsch synthesis. Additionally, the occlusivity agent may include silicone waxes, polymethylsiloxane alkyls, alkoxys and/or esters.

Additional Optional Components

The formulation may also contain a number of optional ingredients. In particular, these optional components are selected from those known in the art to be ingredients used in personal care or pharmaceutical formulations. Illustrative, non-limiting examples include surfactants, solvents, powders, coloring agents, thickeners, waxes, gelling agents or clays, stabilizing agents, pH regulators, silicones, or other suitable agents.

Thickening agent may be added to provide a desired or convenient viscosity. For example, viscosities within the range of 500 to 25,000 mm²/s at 25° C. Alternatively, thickening agents may be added to obtain viscosities within the range of about 3,000 to about 7,000 mm²/s. Suitable thickening agents are exemplified by sodium alginate, gum arable, polyoxyethylene, guar gum, hydroxypropyl guar gum, ethoxylated alcohols, such as laureth-4 or polyethylene glycol 400, cellulose derivatives exemplified by methylcellulose, methylhydroxypropylcellulose, hydroxypropylcellulose, polypropylhydroxyethylcellulose, starch, and starch derivatives exemplified by hydroxyethylamylose and starch amylose, locust bean gum, electrolytes exemplified by sodium chloride and ammonium chloride, and saccharides such as fructose and glucose, and derivatives of saccharides such as PEG-120 methyl glucose diolate or mixtures of 2 or more of these. Alternatively the thickening agent is selected from cellulose derivatives, saccharide derivatives, and electrolytes, or from a combination of two or more of the above thickening agents exemplified by a combination of a cellulose derivative and any electrolyte, and a starch derivative and any electrolyte. The thickening agent may be present in an amount from about 0.05 to about 10% by weight, or, alternatively about 0.05 to about 5% by weight based on the total weight of the formulation.

Also, various cosmetic, personal care, and cosmetic components may be included aside from the excipient or excipients. Examples of suitable cosmetic, and personal care components include, but are not limited to, alcohols, fatty alcohols and polyols, aldehydes, alkanolamines, alkoxylated alcohols butylene copolymers, carbohydrates (e.g. polysaccharides, chitosan and derivatives), carboxylic acids, carbomers, esters, ethers and polymeric ethers (e.g. PEG derivatives, PPG derivatives), glyceryl esters and derivatives, halogen compounds, heterocyclic compounds including salts, hydrophilic colloids and derivatives including salts and gums (e.g. cellulose derivatives, gelatin, xanthan gum, natural gums), imidazolines, inorganic materials (clay, TiO₂, ZnO), ketones (e.g. camphor), isethionates, lanolin and derivatives, organic salts, phenols including salts phosphorus compounds (e.g. phosphate derivatives), polyacrylates and acrylate copolymers, synthetic polymers including salts, siloxanes and silanes, sorbitan derivatives, sterols, sulfonic acids and derivatives and waxes.

Other additives can include powders and pigments. The powder component that may be included can be generally defined as dry, particulate matter having an average particle size of about 0.02-50 microns. The particulate matter may be colored or non-colored (for example, white). Suitable powders include, but are not limited to, bismuth oxychloride, titanated mica, fumed silica, spherical silica beads, polymethylmethacrylate beads. The above mentioned powders may be surface treated to render the particles hydrophobic in nature.

The powder component also may also include various organic and inorganic pigments. The organic pigments are generally various aromatic types including azo, indigoid, triphenylmethane, anthraquinone, and xanthine dyes. Inorganic pigments generally consist of insoluble metallic salts of certified color additives, referred to as the Lakes or iron oxides. A pulverulent coloring agent, such as carbon black, and titanium dioxide, pearlescent agents, generally used as a mixture with colored pigments, or some organic dyes, generally used as a mixture with colored pigments and commonly used in the cosmetics industry, can be added to the formulation. In general, these coloring agents can be present in an amount by weight from about 0 to 20% with respect to the weight of the final formulation.

Pulverulent inorganic or organic fillers can also be added, generally in an amount by weight from about 0 to about 40% with respect to the weight of the final formulation. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked, copolymer microspheres, polytrap, and silicone resin microbeads.

Optional components included in the present formulation may also include other silicones (including any already described above), organofunctional siloxanes, alkylmethylsiloxanes, siloxane resins and silicone gums.

The topical formulations according to the present disclosure may be in the form of a cream, a gel, a powder, a paste, or a freely pourable liquid. Generally, such formulations can generally be prepared at room temperature if no solid materials at room temperature are presents in the formulations, using simple propeller mixers, Brookfield counter-rotating mixers, or homogenizing mixers. No special equipment or processing conditions are typically required. Depending on the type of form made, the method of preparation will be different, but such methods are well known by those of ordinary skill in the art.

If the formulation is prepared without water, an anhydrous formulation results. Such formulations that do not include water may be prepared without the addition of any preservatives.

In embodiments where the substrate is skin, the formulation is applied to the skin to deliver the active agent to the skin. The skin may be healthy and intact, or it may be damaged or wounded. The formulation may be applied, i.e., rubbed or coated, directly onto the skin. Alternatively, the formulation may be deposited on a transdermal patch prior to application of the formulation to the substrate, i.e., to the skin

The controlled-release formulation according to the present disclosure is capable of delivering performance properties such as controlled tack, controlled lubrication, water resistance, and barrier properties. This controlled-release formulation has substantivity to the skin and other substrates, such as teeth. The significant substantivity of the formulation is particularly advantageous when a controlled rate of delivery of the active agent is required over an extended period of time. Simply stated, the controlled-release formulation is topically applied to the substrate where the film remains over the extended period of time, which may be four hours or longer, or eight hours or longer. When the substrate is skin, the substantivity is important due to the presence of certain body oils and especially upon application to skin covered with hair. The formulation also has substantivity to wet substrates such as gums, teeth and mucosal membrane.

The formulations according to the present disclosure can be used by standard and well-known methods, such as applying them to the human body, e.g. skin, hair, or teeth, using applicators, brushes, applying by hand, pouring them and/or possibly rubbing or massaging the formulation onto or into the body. Removal methods are also well known standard methods, including washing, wiping, peeling and the like. According to some embodiments, no removal of the formulation is required as the formulation is fully absorbed into the skin, such that no residue remains on the skin. An effective amount of the formulation for the particular purpose is applied to the skin. Such effective or therapeutic amounts generally range from about 1 mg/cm² to about 10 mg/cm². Application to the skin typically includes working the formulation into the skin. This method for applying to the skin comprises the steps of contacting the skin with the formulation in an effective amount and then rubbing the formulation onto the skin. These steps can be repeated as many times as desired to achieve the desired benefit.

### Examples

These examples are intended to illustrate the invention to one of ordinary skill in the art and should not be interpreted as limiting the scope of the invention set forth in the claims. All measurements and experiments were conducted at 25° C., unless indicated otherwise.

As used herein, “Carbopol® 971P NF” is a polyacrylic acid (Lubrizol Advanced Materials, Lubrizol Corporation (Cleveland, Ohio)). “CLP” is clobetasol propionate, USP grade (Spectrum Chemical Mfg. Corp. (New Brunswick, N.J.)). “Clobetasol propionate 0.05% USP ointment” is a topical ointment containing 0.05% clobetasol propionate (E. Fougera & Co., a division of Nycomed U.S. Inc. (Melville, N.Y.)). “Cosmetic Wax” is a cosmetic wax including stearyl dimethicone (and) octadecene (Dow Corning Corporation (Midland, Mich.)). “DCF” is diclofenac sodium, USP grade (Spectrum Chemical Mfg. Corp. (New Brunswick, N.J.)). “Eudragit® E100” is poly(butyl methacrylate-co-(2-dimethylaminoethyl) methacrylate-co-methyl methacrylate) 1:2:1 (Evonik Industries (Parsippany, N.J.)). “Eudragit® S100” is a poly(methacrylic acid-co-methyl methacrylate) 1:2, (Evonik Industries (Parsippany, N.J.)). “Eudragit® L100” is a poly(methacrylic acid-co-methyl methacrylate) 1:1 (Evonik Industries (Parsippany, N.J.)). “Eudragit® L100-55” is a poly(methacrylic acid-co-ethyl acrylate) 1:1 (Evonik Industries (Parsippany, N.J.)). “HCO” is hydrocortisone, USP grade (Sigma-Aldrich Co. (St. Louis, Mo.). “HMDS” is hexamethyldisiloxane, (Dow Corning Corporation (Midland, Mich.)). “Hydrocortisone 0.5% cream” is a topical cream containing 0.5% hydrocortisone (Walgreen Co. (Deerfield, Ill.)). “IBP” is ibuprofen, USP grade (Spectrum Mfg. Corp. (New Brunswick, N.J.)). “Ibutop 5%” is a topical gel containing 5% Ibuprofen (Dolorgiet GmbH & Co. KG (Bonn, Germany)). “IPA” is isopropyl alcohol, HPLC grade (Fisher Scientific (Fair Lawn, N.J.)) “OLAC” is oleic acid, NF/FCC grade (Fisher Scientific (Fair Lawn, N.J.)). “Petrolatum” is from Spectrum Chemicals Mfg. Corp. (New Brunswick, N.J.). “PG” is propylene glycol, USP/FCC grade (Fisher Scientific (Fair Lawn, N.J.)). “SEB1” is a silicone organic elastomer blend of isododecane and dimethicone/bis-isobutyl propylene glycol 20 cross polymer with 15% solids (Dow Corning Corporation (Midland, Mich.)). “SEB2” is a silicone elastomer blend of cyclopentasiloxane and dimethicone cross polymer with 12.4% solids (Dow Corning Corporation (Midland, Mich.)). “SGM” is a silicone gum containing hydroxyl-terminated dimethyl siloxane (Dow Corning Corporation (Midland, Mich.)). “Voltaren® Gel” is a topical gel containing 1% diclofenac sodium (Novartis Consumer Health Inc. (Parsippany, N.J.)).

### Examples 1-3A

Formulation Ex. 1 was prepared by weighing 0.1590 g of IBP in a speed mixer cup followed by the addition of 0.3158 g of PG, 0.0351 g of OLAC, and 0.6514 g of IPA. The speed mixer cup was closed with a lid and was gently hand-rotated (shaken) until the IBP was completely dissolved. To this, 2.0054 g of the silicone elastomer blend SEB1 (with 26.2% solids content) was weighed into the speed mixer cup, the speed mixer cup was closed with lid and the contents were mixed in the speed mixer until a uniform, homogeneous material was obtained. The formulation material was mixed using a spatula in between the speed mixer mixing cycles to achieve the homogeneous formulation. The SEB1 silicone elastomer blend contains silicone elastomer material blended with isododecane with a solid content of about 15%. Prior to the preparation of the formulation, the SEB1 silicone elastomer blend was concentrated to obtain the 26.2% solids content by evaporating the isododecane from the SEB1 silicone elastomer blend by keeping the material in the oven at 100° C. Gravimetric determination was carried out during the evaporation process to reach the 26.2% solid content.

Formulation Exs. 2 and 3 were prepared using the 26.2% solids elastomer blend following a similar procedure to that described above by changing the amount of individual components as shown below in Table 1.

Formulation 3A was prepared by following the above procedure using the SEB2 silicone elastomer blend (with 26% solids content). Prior to the preparation of the formulation, the SEB2 silicone elastomer blend was concentrated to get the 26% solids content similar to that carried out for SEB1 silicone elastomer blend as described above. The composition of formulation Ex. 3A is shown in Table 1 below.

TABLE 1
Composition of Formulation Examples 1-3 and 3A.

| | Formulation examples | | | |
|---|---|---|---|---|
| | 1 | 2 | 3 | 3A |
| Ingredients | % (w/w) | | | |
| Silicone organic elastomer material | 16.6 | 15.0 | 15.8 | — |
| (from SEB1) | | | | |
| Isododecane (from SEB1) | 46.8 | 42.4 | 44.5 | — |
| Silicone elastomer material (from SEB2) | — | — | — | 18.5 |
| Cyclopentasiloxane (from SEB2) | — | — | — | 52.7 |
| PG | 10.0 | 9.1 | 13.5 | 6.4 |
| OLAC | 1.1 | 1.0 | 1.5 | 0.7 |
| IPA | 20.6 | 27.5 | 19.7 | 16.7 |
| IBP | 5.0 | 5.0 | 5.0 | 5.0 |
| Total | 100.0 | 100.0 | 100.0 | 100.0 |

The permeability behavior, the flux, or the amount of ibuprofen delivered through skin per unit area per unit time, (μg/cm²/hr) from the above formulations was determined using Franz cell permeability experiment set-up at 32° C. and using the epidermal layer of human cadaver skin. In the Franz cell set-up, initially the bottom compartment of a cell was placed in the unit and filled with 3 mL of phosphate buffered saline (PBS, pH 7.4). A small magnetic stir bar was added to the cell. The permeation area in the Franz cell was 0.63 cm². The thawed epidermal layer of skin membrane (as a circle, 1.5875 cm diameter, 1.98 cm² area) was then carefully transferred to the top of the bottom compartment. For each formulation, 3 cells (triplicate) were prepared. About 20 mg of the formulation was taken using positive displacement pipette, applied on the skin and spread manually to achieve a visibly homogeneous distribution. The top compartment (cap) of the Franz cell was attached to the top of the skin and both the top and bottom compartments were clamped together. PBS was added to an appropriate volume of the cell, about 5 mL, and then the permeability experiment was started. The experiment was carried out for 8 hours. During the 8 hour period, 1 mL of sample was collected from the bottom compartment and replaced with fresh PBS solution at 0.5, 1, 2, 4, and 6 hours. At 8 hours, 1 mL of sample was collected. All the samples collected were taken for ultra performance liquid chromatography (UPLC) analysis to determine the ibuprofen concentration using appropriate UPLC method. The benchmark (Ibutop 5%) was used in each set of permeability experiments carried out for the test formulations 1-21.

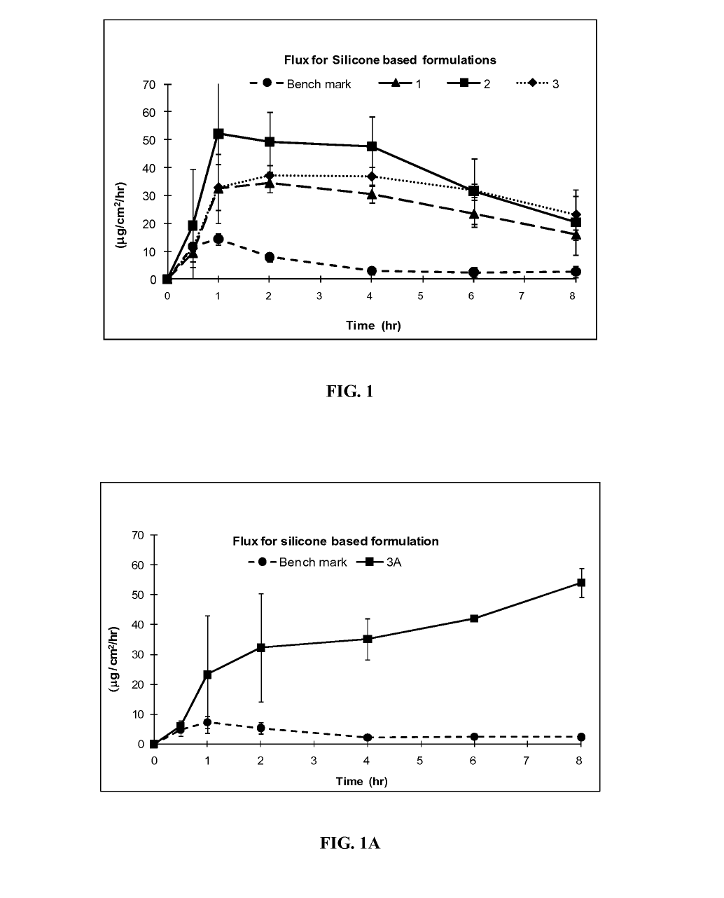

The flux profile for the formulation Exs. 1-3 is provided in FIG. 1. FIG. 1 also shows the flux profile for the Ibutop 5% benchmark, applied in the same amount, 20 mg, to the same amount (area) of the skin membrane. The flux experiment was carried out at the same time using the same conditions for all the formulations and for the benchmark. The flux profile for formulation Ex. 3A and the Ibutop 5% benchmark is shown in FIG. 1A.

As seen in FIGS. 1-7, the commercially available benchmark product containing 5% by weight of Ibuprofen delivers less than about 8 μg/cm²/hr to the skin membrane after 2 hours and less than about 5 μg/cm²/hr to the skin membrane after 4 hours. Moreover, as can be seen from Table 5 below, the benchmark delivers a cumulative amount of between about 13 and 23.5 μg after 8 hours, which represents only between about 1.33% and 2.35% by weight of the drug that is present in the benchmark. The benchmark exhibited a maximum flux at about 13 μg/cm²/hr about 1 hour after application to the membrane. After about 1 hour, the amount of drug delivered by the benchmark significantly decreased and demonstrated little to no sustained release over the 8 hour test period. A burst effect is generally characterized by an increase in the flux value over a short period of time and hence the release shown by the benchmark may be considered as a burst effect. After about 4 hours, the benchmark delivered a very small amount of the drug which may provide negligible therapeutic effect.

As seen in FIG. 1, the 5% Ibuprofen formulation prepared in Ex. 2 has the highest flux profile out of Exs. 1, 2, and 3. The flux of all the formulations prepared in Exs. 1, 2, and 3 is significantly higher than that of the Ibutop 5% benchmark. After 1 hour, the formulations exhibited a significant burst effect. The formulation prepared in Ex. 2 had the strongest burst effect, with a flux being over 50 μg/cm²/hr after 1 hour.

The formulation prepared in Ex. 2 had a flux of slightly below 50 μg/cm²/hr 2-4 hours after application, a flux of about 35 μg/cm²/hr 6 hours after application, and a flux of about 25 μg/cm²/hr 8 hours after application. As seen in Table 5 below, the application of the formulation prepared in Ex. 2 to the skin resulted in about 180 μg of IBP being delivered to the skin after 8 hours, which is about 8 times higher than the benchmark. Moreover, about 18% of the drug present in the formulation prepared in Ex. 2 was delivered to the skin after 8 hours, which is also about 8 times higher than the benchmark.

The formulation prepared in Ex. 1 had a flux of about 30 μg/cm²/hr 2-4 hours after application, a flux of about 25 μg/cm²/hr 6 hours after application, and a flux of about 20 μg/cm²/hr 8 hours after application. As seen in Table 5 below, the application of the formulation prepared in Ex. 1 to the skin resulted in about 124 μg of IBP being delivered to the skin after 8 hours, which is about 5 times higher than the benchmark. Moreover, about 12% of the drug present in the formulation prepared in Ex. 1 was delivered to the skin after 8 hours, which is also about 5 times higher than the benchmark.

The formulation prepared in Ex. 3 had a flux of about 35 μg/cm²/hr 2-6 hours after application and a flux of about 30 μg/cm²/hr 8 hours after application. The formulations prepared in Exs. 1-3 can provide a therapeutic effect, generally, pain relief for ibuprofen, for at least eight hours or more. Additionally, due to the significantly higher flux of the formulations prepared in Exs. 1-3 as opposed to the benchmark, a significantly lower amount of active ingredient needs to be used to achieve a therapeutic effect provided by 5% drug in benchmark, which will be further shown in by Exs. 40-42 below. As seen in Table 5 below, the application of the formulation prepared in Ex. 3 to the skin resulted in about 154 μg of IBP being delivered to the skin after 8 hours, which is about 6.5 times higher than the benchmark. Moreover, about 15% of the drug present in the formulation prepared in Ex. 3 was delivered to the skin after 8 hours, which is also about 6.5 times higher than the benchmark.

As seen in FIG. 1A, the formulation prepared in Ex. 3A had a flux of about 22 μg/cm²/hr after 1 hour. After 2-4 hours, the flux was about 30 μg/cm²/hr. After 6 hours, the flux increased to about 35 μg/cm²/hr. Finally, after 8 hours, the flux was the highest out of all the measurements, with a value of about 50 μg/cm²/hr. Therefore, formulation prepared in Ex. 3A is particularly well-suited for applications that require extended release of higher amount of active ingredient into the skin. The formulation prepared in Ex. 3A exhibited a burst effect about 1 hour after application and had sustained release up to 8 hours following application. As seen in Table 5, the application of the formulation prepared in Ex. 3A to the skin resulted in about 197 μg of IBP being delivered to the skin after 8 hours, which is about 12 times higher than the benchmark. Moreover, about 20% of the drug present in the formulation prepared in Ex. 3A was delivered to the skin after 8 hours, which is also about 12 times higher than the benchmark.

Thus, the silicone elastomer blend containing formulations prepared in Exs. 1-3A exhibited a significantly better flux profile than the Ibutop benchmark. Moreover, the application of the formulations prepared in Exs. 1-3A to the skin resulted in significantly larger amounts of the drug actually being delivered to the skin after a period of time. As seen in Table 5, the application of the Ibutop benchmark to Donor 1's tissue of Donor 1 resulted in only about 2.35% by weight of the drug actually being delivered to the skin and the application of the Ibutop benchmark to Donor 2's tissue resulted in only about 1.62% by weight of the drug actually being delivered to the skin after 8 hours. The application of the formulations prepared according to Exs. 1-3A resulted in about 5-12 times more drug actually being delivered to the skin than the benchmark. The formulations prepared in Exs. 1-3A result in a much more economical and efficient product since a significantly higher percentage of the drug actually gets delivered to the skin.

### Examples 4-21

Formulation Exs. 4-21 were prepared using commonly used non-silicone based excipients in topical formulations, petrolatum, Carbopol® and acrylic polymers, in place of silicone excipients used in Exs. 1-3A above. Other excipients, PG, OLAC, IPA, were used as in formulation Exs. 1-3A to achieve similar formulations. The flux profile of the resulting formulations (4-21) was tested and compared for efficiency of delivering IBP through the skin with the silicone formulation Exs. 1-3A. Silicone formulation Exs. 1-3A delivered a higher amount of the drug at 1 hr than the benchmark and than formulation Exs. 4-21. Moreover, the silicone formulation Exs. 1-3A also released a higher amount of the drug after 8 hours.

### Examples 4-6

Formulation Ex. 4 was prepared by weighing 3.0050 g of petrolatum in a speed mixer cup followed by the addition of 0.5413 g of PG, 0.0601 g of OLAC and mixed in the speed mixer for homogeneity. 0.1897 g of ibuprofen was then weighed, added to the speed mixer and mixed again until the drug completely dissolved. For formulation Exs. 5 and 6, an appropriate amount of IPA was also added (see Table 2) after adding IBP. The formulation was mixed using a spatula in between the speed mixer mixing cycles to achieve a homogeneous formulation.

Similar to that mentioned for the formulation Exs., 1, 2, and 3, the flux experiment was carried out for formulation Exs. 4, 5 and 6. FIG. 3 shows the flux profile for formulation Exs. 4, 5, and 6 along with the flux profile for commercially available benchmark product (Ibutop 5% gel). The flux experiment was carried out at the same time using the same conditions for all the formulations and the bench mark. About 20 mg of the formulations prepared in Exs. 4-6 was applied to the epidermis of Donor 3.

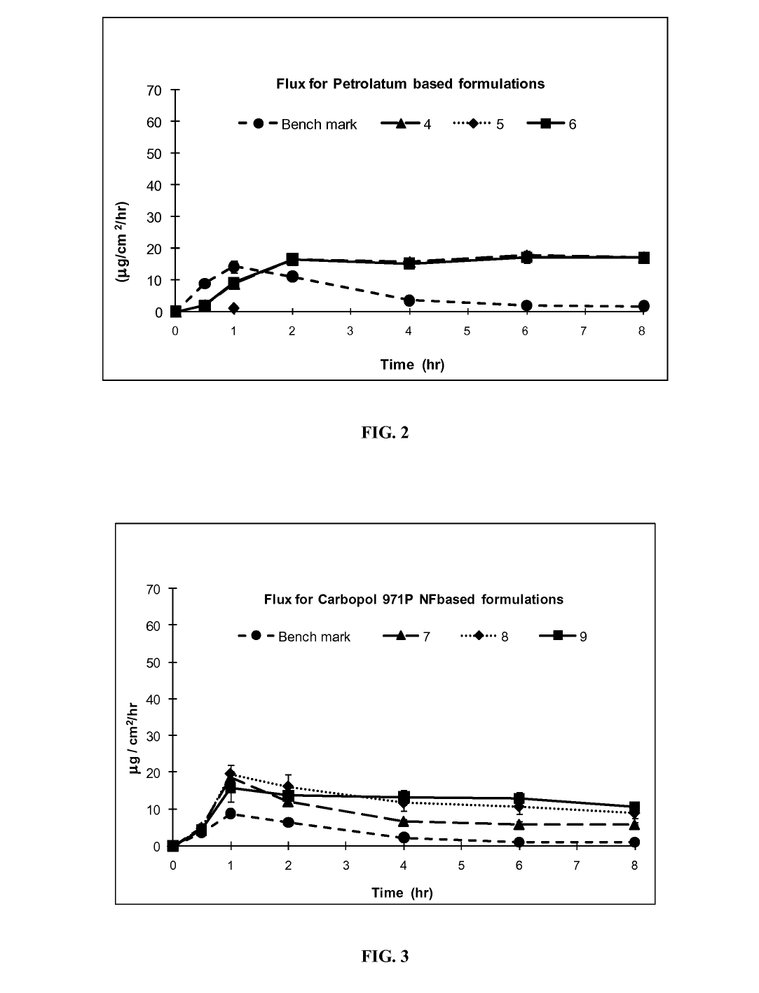

As seen in FIG. 2, the petrolatum based formulations prepared in Exs. 4-6 did not exhibit a burst effect. After 1 hour, those formulations had a flux profile of about 8 μg/cm²/hr. The flux increased to about 15 μg/cm²/hr 2 hours after application and remained at that value until the end of the experiment, which occurred 8 hours after the application. The petrolatum based formulations had a higher flux than the benchmark but a significantly lower flux than the formulations prepared in Exs. 1-3A. As seen in Table 5, after 8 hours, the cumulative release to the skin from the formulations prepared in Exs. 4-6 was about 80 μg and about 8% by weight of the drug was delivered to the skin, whereas silicone based formulation Exs. 1-3A showed a cumulative release of about 124-196 μg in the same time period. The amount delivered at 1 hr by petrolatum based formulations was lower than that of the benchmark and the silicone based formulations.

TABLE 2
Composition of Formulation Examples 4-6.

| | Formulation examples | | |
|---|---|---|---|
| | 4 | 5 | 6 |
| Ingredients | % (w/w) | | |
| Petrolatum | 79.2 | 81.3 | 74.8 |
| PG | 14.3 | 9.8 | 13.5 |
| OLAC | 1.6 | 1.1 | 1.5 |
| IPA | 0.0 | 2.9 | 5.2 |
| IBP | 5.0 | 5.0 | 5.0 |
| Total | 100.0 | 100.0 | 100.0 |

### Examples 7-9

Formulation Ex. 7 was prepared by weighing 0.2017 g of Carbopol® 971P NF in a scintillation vial followed by the addition of 3.5040 g of IPA. The mixture was mixed in a vortex mixer followed by the addition of 1.5078 g of water. After the addition of water, it was mixed again in the vortex mixer. To the vial, 0.0941 g of PG, 0.0105 g of OLAC and 0.2796 g of IBP were added and mixed using the vortex mixer to obtain a homogeneous clear formulation in which the ibuprofen was completely dissolved. Similar procedure was followed to prepare the formulation Exs. 8 and 9 by changing the amount of individual components as shown in Table 3 below.

Similar to the formulation Exs., 1, 2, and 3, the flux experiment was carried out for Exs. 7, 8, 9, and the benchmark. FIG. 3 shows the flux profile for formulation Exs. 7, 8, and 9 along with that for the benchmark (Ibutop 5% gel). The flux experiment was carried out at the same time using the same conditions for all the formulations and the benchmark. About 20 mg of the formulation prepared in Exs. 7-9 was applied to the epidermis of Donor 4.

TABLE 3
Composition of Formulation Examples 7-9.

| | Formulation examples | | |
|---|---|---|---|
| | 7 | 8 | 9 |
| Ingredients | % (w/w) | | |
| Carbopol ® 971P NF | 3.6 | 3.5 | 3.4 |
| Water | 26.9 | 26.0 | 25.0 |
| PG | 1.7 | 4.7 | 7.5 |
| OLAC | 0.2 | 0.5 | 0.8 |
| IPA | 62.6 | 60.4 | 58.3 |
| IBP | 5.0 | 5.0 | 5.0 |
| Total | 100.0 | 100.0 | 100.0 |

As seen from FIG. 3, the Carbopol® 971P NF based formulations prepared in Exs. 7-9 exhibit a slightly better flux profile than the benchmark. Unlike the benchmark, these formulations exhibit an initial burst effect and provide about 17-20 μg/cm²/hr flux 1 hour after application. However, during the time period falling between about 2-8 hours after application, these formulations exhibit a flux profile of about 9-15 μg/cm²/hr, with the formulation prepared in Ex. 7 exhibiting the lowest flux profile and the formulation prepared in Ex. 8 exhibiting the highest flux profile. The formulation prepared in Ex. 9 had the steadiest flux profile, with the flux remaining at about 15 μg/cm²/hr between 1-8 hours after application. As seen in Table 5, the Carbopol® 971P NF based formulations resulted in between about 39 and 62 μg, or about 3.9 and 6.2% by weight of the drug being delivered to the skin after 8 hours whereas silicone based formulation Exs. 1-3A showed a cumulative release of about 124-196 μg in the same time period. The amount delivered at 1 hr by Carbopol® based formulations was lower than that of the silicone based formulations prepared in Exs. 1-3A.

### Examples 10-21

For the preparation of formulation Exs. 10, 11, and 12, initially an about 50% solids stock solution of Eudragit® E100 was made by dissolving it in a predetermined amount of IPA to achieve 50% solids. Formulation Ex. 10 was prepared by weighing 4.0142 g of the above 50% solids solution of Eudragit® E100 in a scintillation vial followed by the addition of 0.9196 g of PG, 0.1022 g of OLAC and 0.2565 g of IBP. The mixture was mixed in a vortex mixer to get a homogeneous clear formulation in which the ibuprofen was completely dissolved. Similar procedure was followed to prepare Exs. 11 and 12 by changing the amount of individual components as shown in Table 4 below.

For the preparation of Exs. 13, 14, and 15, initially an about 25% solids stock solution of Eudragit® S100 was made by dissolving it in a predetermined amount of IPA to achieve a 25% solids content. This stock solution was used to make the formulations following the procedure above for the preparation of formulation Ex. 10 as shown in Table 4 below.

For the preparation of Exs. 16, 17, and 18, initially a 25% solids stock solution of Eudragit® L100 was made by dissolving it in an appropriate amount of IPA to achieve a 25% solids content. This stock solution was used to make the formulation following the procedure mentioned above for the preparation of formulation Ex. 10 as shown in Table 4 below.

For the preparation of formulation Exs. 19, 20, and 21, initially a 25% solids stock solution of Eudragit® L100-55 was made by dissolving it in an appropriate amount of IPA to achieve a 25% solids. This stock solution was used to make the formulation following the procedure mentioned above for the preparation of formulation Ex. 10 content as shown in Table 4 below.

The compositions of all the formulations, 10-21 are shown below in Table 4.

TABLE 4
Composition of Formulation Examples 10-21.

| | Formulation examples | | | | | | | | | | | |
|---|---|---|---|---|---|---|---|---|---|---|---|---|
| | 10 | 11 | 12 | 13 | 14 | 15 | 16 | 17 | 18 | 19 | 20 | 21 |
| Ingredients | % (w/w) | | | | | | | | | | | |
| Eudragit ® E100 | 39.1 | 35.3 | 30.9 | | | | | | | | | |
| Eudragit ® S100 | | | | 20.6 | 17.8 | 16.2 | | | | | | |
| Eudragit ® L100 | | | | | | | 21.3 | 19.8 | 18.0 | | | |
| Eudragit ® L100-55 | | | | | | | | | | 21.7 | 19.7 | 18.0 |
| PG | 17.9 | 23.8 | 31.1 | 18.9 | 24.2 | 29.3 | 11.7 | 17.8 | 22.8 | 11.8 | 17.6 | 23.0 |
| OLAC | 2.0 | 2.6 | 3.5 | 2.1 | 2.7 | 3.3 | 1.3 | 2.0 | 2.5 | 1.3 | 2.0 | 2.6 |
| IPA | 36.1 | 33.2 | 29.6 | 53.4 | 50.3 | 46.3 | 60.7 | 55.4 | 51.7 | 60.1 | 55.7 | 51.4 |
| IBP | 5.0 | 5.0 | 5.0 | 5.0 | 5.0 | 5.0 | 5.0 | 5.0 | 5.0 | 5.0 | 5.0 | 5.0 |
| Total | 100.0 | 100.0 | 100.0 | 100.0 | 100.0 | 100.0 | 100.0 | 100.0 | 100.0 | 100.0 | 100.0 | 100.0 |

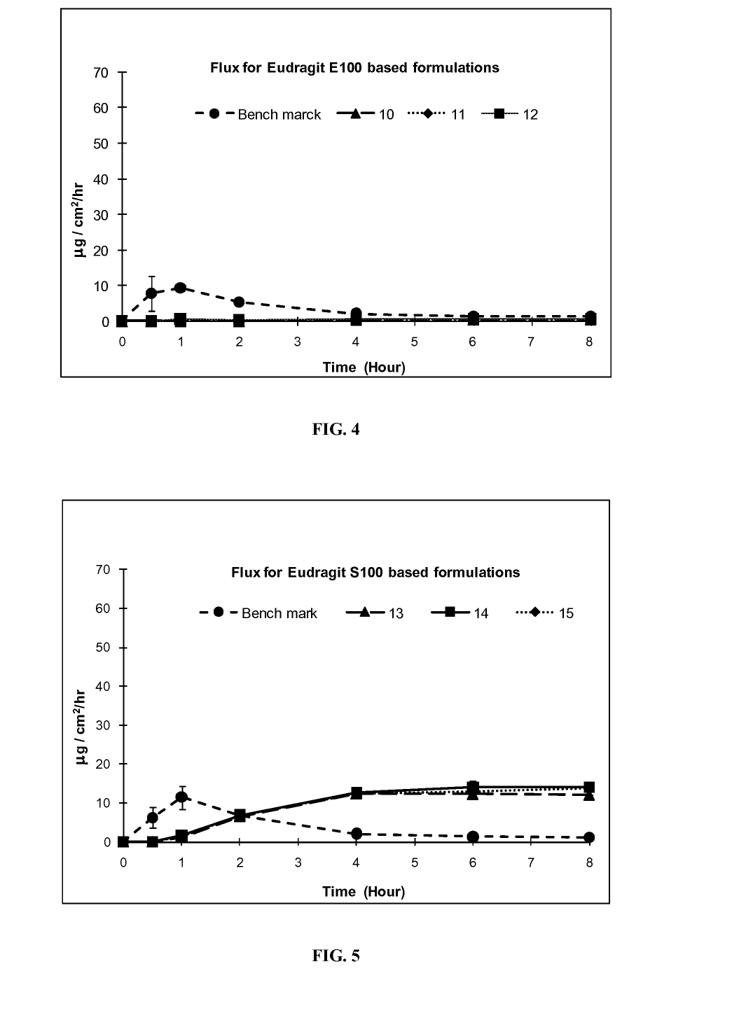

Similar to that carried out for formulation Exs., 1, 2, and 3, the flux experiment was carried out for Exs. 10-21 and the benchmark. FIGS. 4-7 show the flux profile for formulation Exs. 10-21 along with that for the benchmark (Ibutop 5% gel). The flux experiment was carried out at the same time using the same conditions for all the formulations and the bench mark. About 20 mg of the formulations prepared in Exs. 10-21 was applied to the membrane.

As seen in FIG. 4, the flux profile of the Eudragit® E100 based formulations prepared in Exs. 10-12 is actually worse than that for the benchmark. The Eudragit® E100 based formulations allow very little, if any, flux through the skin. As seen in Table 5, the Eudragit® E100 based formulations resulted in between about 2 and 2.5 μg, or about 0.2 and 0.25% by weight of the drug being delivered to the skin after 8 hours. As discussed above, the silicone based formulations prepared in Exs. 1-3A resulted in cumulative release of about 124-196 μg, or about 12.4-19.6% by weight, of IBP after 8 hours. Thus, the silicone based formulations prepared in Exs. 1-3A delivered about 50-100 times more IBP than the Eudragit® E100 based formulations prepared in Exs. 10-12.

As seen in FIG. 5, the flux profile of the Eudragit® S100 based formulations prepared in Exs. 13-15 is slightly better than that of the benchmark. Unlike silicone based formulations or the benchmark, it took the Eudragit® S100 based formulations almost 2 hours to deliver any significant flux to the skin; between about 2 hours to about 8 hours after application, those formulations delivered about 13 μg/cm²/hr to the skin membrane. As seen in Table 5 the Eudragit® S100 based formulations resulted in between about 52 and 57 μg, or about 5.2 and 5.7% by weight of the drug being delivered to the skin after 8 hours. While the amount released by the Eudragit® S100 based formulations delivered a higher amount of the drug than the benchmark, the Eudragit® S100 based formulations delivered a significantly lower amount of the drug than the silicone elastomer blend based formulations Exs. 1-3A, which delivered a cumulative amount of about 124-196 μg, or about 12.4 to 19.6% by weight, of the drug after 8 hours. In other words, the silicone elastomer blend formulations prepared in Exs. 1-3A delivered about 2.5-4 times more drug than the Eudragit® S100 based formulations

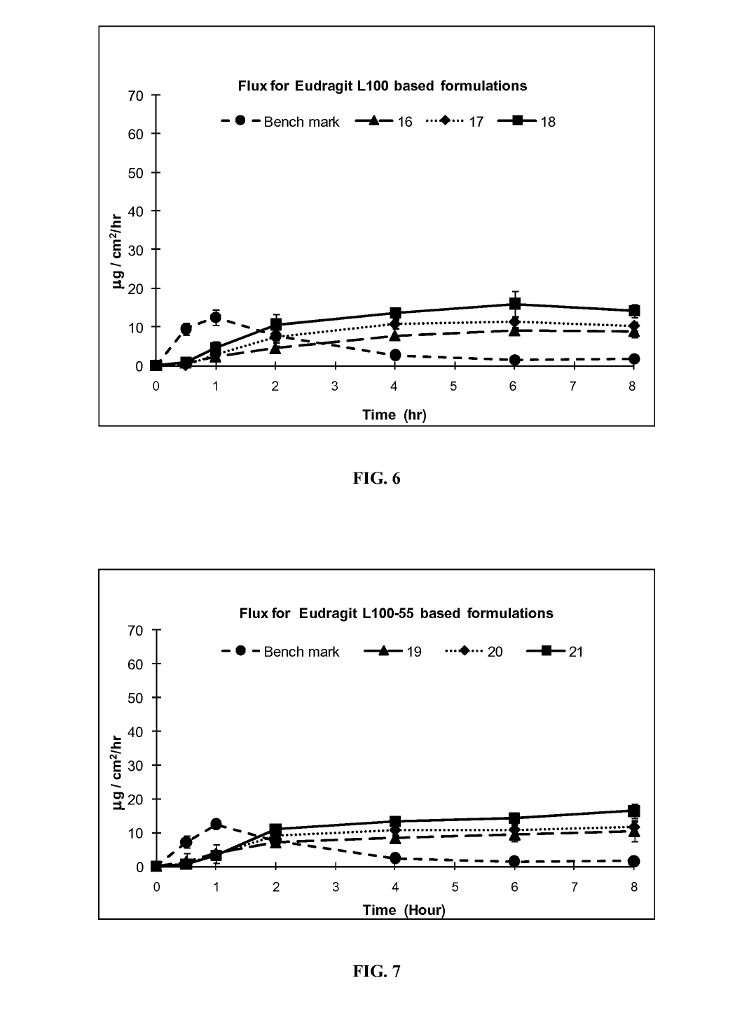

As seen in FIGS. 6 and 7, Eudragit L100 formulations prepared in Exs. 16-18 and Eudragit® L100-55 based formulations prepared in Exs. 19-21 exhibited similar flux profiles to Eudragit® S100 based formulations and delivered between about 10-13 μg/cm²/hr between about 2 hours to about 8 hours after application. As seen in Table 5 below, the Eudragit® L100 and L100-55 based formulations resulted in between about 36 and 64 μg, or about 3.6 and 6.4% by weight of the drug being delivered to the skin after 8 hours. The silicone elastomer blend based formulations prepared in Exs. 1-3A delivered about 124-196 μg, which represents about 12.4 to 19.6% by weight, of IBP after 8 hours. In other words, silicone elastomer blend formulations prepared in Exs. 1-3A delivered about 2 to about 5 times more IBP to the skin after 8 hours than the Eudragit® L100 and L100-55 based formulations.

The Eudragit® polymer based formulations Exs. 13-21 delivered a higher cumulative amount of the drug to the skin after 8 hours than the benchmark. However, those formulations delivered a smaller amount of the drug to the skin than the benchmark after 1 hour. For this particular pain reliever drug (IBP), a quicker release of the drug is more beneficial to the patient to relieve the pain quicker. The silicone based formulations prepared in Exs. 1-3A not only showed a higher release after 1 hr as compared to the benchmark, but also showed a higher cumulative release after 8 hours as compared to the benchmark and formulations of Exs. 13-21.

TABLE 5
Cumulative Amount and Percent Drug Release for Formulation Examples
1-21 and Corresponding Benchmark.

| | Cumulative Amount | Total | | |
|---|---|---|---|---|
| | Delivered | Time | Drug Release | Skin |
| Formulation | (μg) | (hrs) | (% wt.) | Epidermis |
| Benchmark | 23.51 | 8 | 2.35 | Donor_1 |
| 1 | 124.43 | 8 | 12.44 | (FIG. 1) |
| 2 | 180.37 | 8 | 18.04 | |
| 3 | 154.25 | 8 | 15.42 | |
| Benchmark | 16.24 | 8 | 1.62 | Donor_2 |
| 3A | 196.69 | 8 | 19.66 | (FIG. 1A) |
| Benchmark | 23.39 | 8 | 2.33 | Donor_3 |
| 4 | 78.2 | 8 | 7.82 | (FIG. 2) |
| 5 | 83.06 | 8 | 8.3 | |
| 6 | 76.7 | 8 | 7.67 | |
| Benchmark | 13.37 | 8 | 1.33 | Donor_4 |
| 7 | 38.64 | 8 | 3.86 | (FIG. 3) |
| 8 | 57.67 | 8 | 5.76 | |
| 9 | 61.87 | 8 | 6.18 | |
| Benchmark | 14.82 | 8 | 1.48 | Donor_5 |
| 10 | 1.84 | 8 | 0.18 | (FIG. 4 |
| 11 | 1.9 | 8 | 0.19 | |
| 12 | 2.52 | 8 | 0.25 | |
| Benchmark | 15.96 | 8 | 1.59 | Donor_6 |
| 13 | 51.63 | 8 | 5.16 | (FIG. 5) |
| 14 | 56.85 | 8 | 5.68 | |
| 15 | 54.59 | 8 | 5.45 | |
| Benchmark | 19.09 | 8 | 1.9 | Donor_7 |
| 16 | 36.36 | 8 | 3.63 | (FIG. 6) |
| 17 | 46.37 | 8 | 4.63 | |
| 18 | 63.92 | 8 | 6.39 | |
| Benchmark | 17.89 | 8 | 1.79 | Donor_8 |
| 19 | 41.96 | 8 | 4.19 | (FIG. 7) |
| 20 | 49.02 | 8 | 4.9 | |
| 21 | 64.17 | 8 | 6.41 | |

### Examples 22-28

Formulation Ex. 22 was prepared by weighing 0.0397 g of DCF in a speed mixer cup followed by the addition of 0.9193 g of IPA, 0.4528 g of PG and 0.0503 g of OLAC. The cup was closed with a lid and was gently mixed using a vortex mixer until the DCF was completely dissolved. Into the same cup, 2.5076 g of SEB1 with 26.2% solids content was added and the cup was closed with the lid. The cup was mixed in the speed mixer until a homogeneous material was obtained. The formulation material was mixed using spatula in between mixing cycles to achieve the homogeneous formulation. Exs. 23-26 were prepared using a similar procedure to that described above by changing the amount of individual components as shown below in Table 6. Exs. 27 and 28 were prepared in a similar manner, but using SEB2 with 26% solids content.

TABLE 6
Composition of Formulation Examples 22-28.

| | Formulation examples | | | | | | |
|---|---|---|---|---|---|---|---|
| | 22 | 23 | 24 | 25 | 26 | 27 | 28 |
| Ingredients | % (w/w) | | | | | | |
| Silicone elastomer only (from SEB1) | 16.6 | 16.5 | 15.3 | 17.5 | 15.4 | — | — |
| Isododecane only (from SEB1) | 46.6 | 46.6 | 43.2 | 49.3 | 43.3 | — | — |
| Silicone elastomer only (from SEB2) | — | — | — | — | — | 17.4 | 16.8 |
| Cyclopentasiloxane only (from SEB2) | — | — | — | — | — | 49.4 | 47.9 |
| PG | 11.4 | 10.2 | 10.7 | 12.1 | 10.7 | 12.1 | 11.8 |
| OLAC | 1.3 | 2.5 | 1.2 | 1.3 | 1.2 | 1.3 | 1.3 |
| IPA | 23.2 | 23.1 | 21.5 | 18.7 | 21.4 | 18.8 | 13.1 |
| Petrolatum | — | — | 7.0 | — | — | — | — |
| Cosmetic Wax | — | — | — | — | 7.0 | — | 8.0 |
| DCF | 1.0 | 1.0 | 1.0 | 1.0 | 1.0 | 1.0 | 1.0 |
| Total | 100.0 | 100.0 | 100.0 | 100.0 | 100.0 | 100.0 | 100.0 |

The permeability behavior, the flux (or the amount of DCF delivered through skin per unit area per unit time, (μg/cm²/hr)) of the DCF from the above formulation examples was determined using Franz cell permeability experiment set-up at 32° C. using epidermis of human cadaver skin as described earlier. The commercially available benchmark product, Voltaren® Gel, 1% DCF topical gel, was used for comparison.

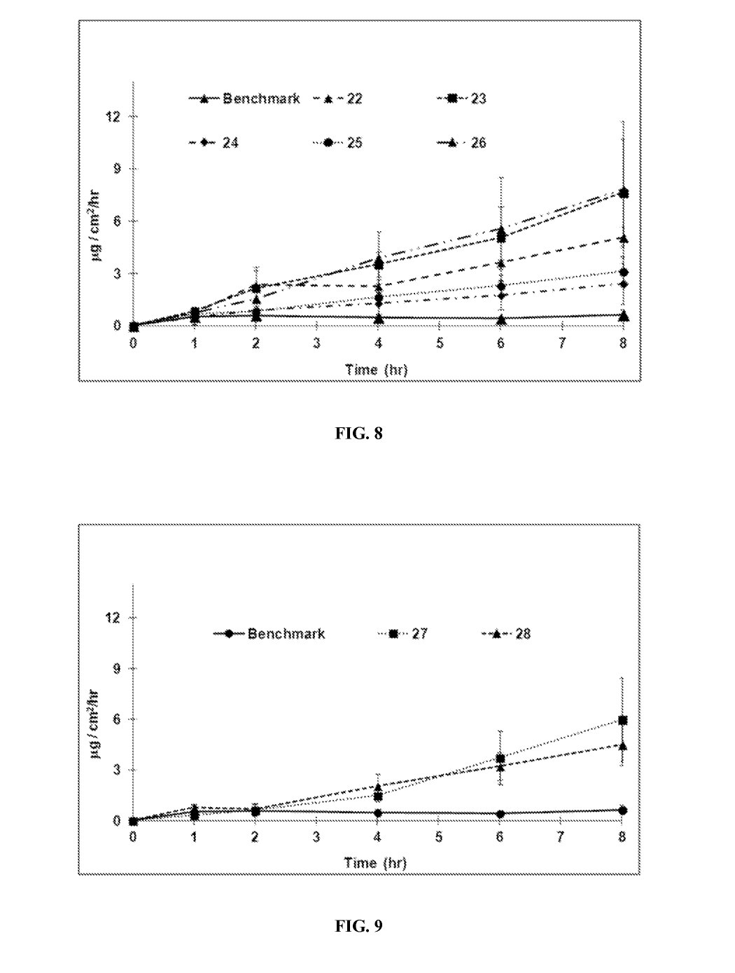

The flux profile for Exs. 22-26 is provided in FIG. 8. FIG. 8 also shows the flux profile for the commercially available benchmark, Voltaren®, applied in the same amount, 20 mg, to the same amount (area) of the skin membrane. The flux experiment was carried out at the same time using the same conditions for all the formulations and for the benchmark. The flux profile for the formulation Exs. 27-28 is provided in FIG. 9. FIG. 9 also shows the flux profile for the benchmark, Voltaren®, applied in the same amount, about 20 mg, to the same amount (area) of the skin membrane.

As seen in FIGS. 8-9, the Voltaren® benchmark delivers less than about 1 μg/cm²/hr to the skin membrane throughout the 8-hour testing period. Moreover, as can be seen from Table 10 below, the benchmark delivered a cumulative amount of about 2.67 μg after 8 hours, which represents only about 1.33% by weight of the drug that is present in the benchmark. The benchmark exhibits a relatively flat flux profile throughout the 8-hour period. Silicone-containing formulations prepared in Exs. 22-28 deliver a significantly higher amount of DCF to the membrane than the benchmark at any point throughout the 8-hour period and cumulatively, as seen in FIGS. 8 and 9 and Table 10. The cumulative release after 8 hours of the formulation prepared in Ex. 26 was about 70 μg, or about 35% by weight, which represents a 26-fold increase over the benchmark product. The formulation prepared in Ex. 24 demonstrated the lowest flux profile out of Exs. 22-28, delivering a cumulative amount of 7.83 μg of DCF, or about 4% by weight, to the membrane after 8 hours—yet, that still represents an almost 3-fold increase over the benchmark. Thus, silicone elastomer blend formulations deliver DCF significantly better than the benchmark.

### Examples 29-31

Formulation Ex. 29 was prepared by weighing 0.0025 g of CLP in a speed mixer cup followed by the addition of 1.4352 g of IPA, 0.4762 g of PG and 0.0529 g of OLAC. The cup was closed with a lid and was gently mixed using a vortex mixer until the CLP was completely dissolved. Into the same cup, 3.0082 g of SEB1 (with 26.2% solids content) was added and the cup was closed with the lid. The cup was mixed in the speed mixer until a homogeneous material was obtained. The formulation material was mixed using a spatula in between the speed mixer mixing cycles to achieve the homogeneous formulation. Formulation ex. 30 was prepared using a similar procedure described above by changing the amount of individual components and using SEB2 with a 26% solids content as shown in Table 7 below.

Formulation Ex. 31 was prepared by weighing 0.2009 g of Carbopol® 971P NF in a speed mixer cup followed by the addition of 3.5088 g of IPA. The mixture was mixed gently in a vortex mixer followed by the addition of 1.5159 g of water. After the addition of water, the contents of the cup were mixed well again in the speed mixer. Into the same cup, 0.4528 g of PG, 0.0503 g of OLAC and 0.0029 g of CLP were added and the contents of the cup were mixed well using the speed mixer to get a homogeneous clear formulation in which the CLP was completely dissolved. The compositions of Exs. 29-31 are shown in Table 7 below.

TABLE 7
Composition of formulation Exs. 29-31.

| | Formulation examples | | |
|---|---|---|---|
| | 29 | 30 | 31 |
| Ingredients | % (w/w) | | |
| Silicone elastomer only (from SEB1) | 15.8 | — | — |
| Isododecane only (from SEB1) | 44.6 | — | — |
| Silicone elastomer only (from SEB2) | — | 19.5 | — |
| Cyclopentasiloxane only (from SEB2) | — | 55.4 | — |
| Carbopol ® 971P NF | — | — | 3.5 |
| PG | 9.6 | 14.6 | 7.9 |
| OLAC | 1.1 | 1.6 | 0.9 |
| IPA | 28.8 | 8.9 | 61.2 |
| Water | — | — | 26.4 |
| CLP | 0.05 | 0.05 | 0.05 |
| Total | 100.0 | 100.0 | 100.0 |

The permeability behavior, the flux (or the amount of CLP delivered through skin per unit area per unit time, (ng/cm²/hr)) of the CLP from the above formulations was determined using Franz cell permeability experiment set-up at 32° C. using epidermis of human cadaver skin as described earlier. The experiment was conducted for a total of 30 hrs. A Clobetasol propionate 0.05% USP ointment benchmark, was used for comparison.

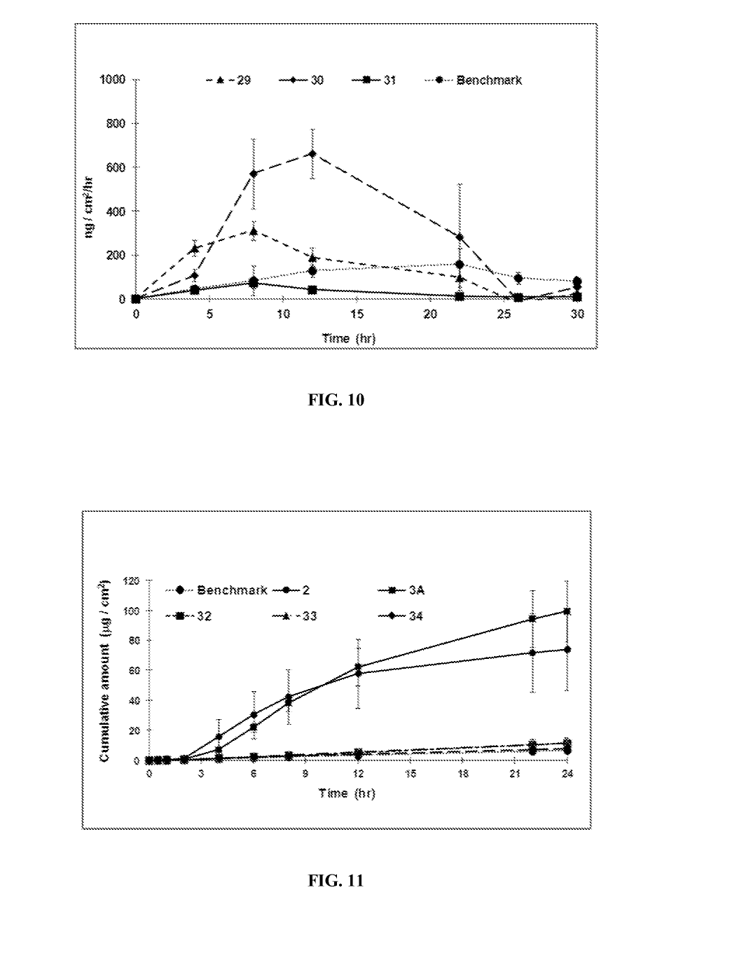

The flux profile for the formulation Exs. 29-31 is provided in FIG. 10. FIG. 10 also shows the flux profile for the commercially available benchmark product (Clobetasol propionate 0.05%) applied in the same amount, 20 mg, to the same amount (area) of the skin membrane. The flux experiment was carried out at the same time using the same conditions for all the formulations and for the benchmark.

As seen in FIG. 10 and Table 10, the silicone-containing formulation examples deliver the CLP significantly better than the commercial benchmark and the formulation Ex. 31 prepared using Carbopol® 971P NF instead of SEB1 or SEB2. Neither the benchmark nor the formulation Ex. 31 exhibited a burst effect. After 30 hours, the commercial benchmark delivered about 2114 ng or 21.14% by weight of CLP to the membrane. The Carbopol® containing formulation Ex. 31 delivered about 518 ng or 5.17% by weight of CLP to the membrane. Formulation Ex. 29 that included SEB1 delivered about 2498 ng or 24.98% by weight of CLP to the membrane, about an 18% improvement over the benchmark. Formulation Ex. 30 that included SEB2 delivered about 5324 ng or 53.24% by weight of CLP to the membrane, a 2.5-fold improvement over the benchmark. Thus, using CLP, silicone elastomer blend containing formulation Exs. 29 and 30 are delivering the drug significantly better than both the benchmark and the formulation Ex. 31 containing Carbopol®.

### Examples 32-34 and 2 and 3A

Formulation Exs. 32-34 were prepared using SGM, as shown below in Table 8. Formulation Ex. 32 was prepared by weighing 0.0506 g of SGM in a scintillation vial followed by the addition of 0.5005 g of IBP and 9.4523 g of HMDS. The vial was closed with a lid and the contents were mixed using vortex mixer. The IBP was not completely dissolved in the resulting solution; instead, it was dispersed in the solution. Exs. 33 and 34 were prepared using a similar procedure to that described above by changing the amount of individual components as shown below in Table 8.

TABLE 8
Composition of formulation examples 32-34.

| | Formulation examples | | |
|---|---|---|---|
| | 32 | 33 | 34 |
| Ingredients | % (w/w) | | |
| SGM 36 | 0.5 | 1.0 | 2.0 |
| HMDS | 94.5 | 94.0 | 93.0 |
| IBP | 5.0 | 5.0 | 5.0 |
| Total | 100.0 | 100.0 | 100.0 |

The cumulative amount of IBP delivered across the skin, (or the total amount of IBP delivered through skin per unit area (μg/cm²) for the entire experimental period of 24 hours) by the formulation Exs. 32-34 was determined using Franz cell permeability experiment set-up at 32° C. using epidermis of human cadaver skin as described earlier. Silicone elastomer blend based formulation Exs. 2 and 3A, prepared as described above in the section entitled “Examples 1-3A,” and the Ibutop benchmark were also included in the experiment.

The flux profile showing the cumulative amount of the drug delivered to the membrane during the 24 hour experiment for the formulation Exs. 32-34, 2, and 3A is provided in FIG. 11. FIG. 11 also shows the cumulative amount of the drug delivered during different stages for the Ibutop benchmark, applied in the same amount, to the same amount (area) of the skin membrane. The permeability experiment was carried out at the same time using the same conditions for all the formulations and for the benchmark. 10 mg of each formulation and the benchmark was applied to the skin of Donor 11 (see Table 10 below).

As seen in FIG. 11 and Table 10, the silicone elastomer blend containing formulations 2 and 3A deliver the IBP to the membrane significantly better than the SGM containing formulation Exs. 32-34 and the commercial benchmark Ibutop. After 24 hours, the commercial benchmark delivered about 4 μg or 1.17% by weight of IBP to the membrane. SGM containing formulation Exs. 32 and 33 delivered about 7 μg or about 1.37% by weight of the drug to the membrane and formulation Ex. 34 delivered about 5 μg or about 0.95% by weight of the drug to the membrane. SEB1 containing formulation Ex. 2 delivered about 47 μg or about 9.9% by weight of the drug to the membrane, an 8.5-fold improvement over the benchmark and a more than 7-fold improvement over the SGM containing formulation Exs. 32-34. SEB2 containing formulation Ex. 3A delivered about 63 μg or about 11.58% by weight of IBP to the membrane, a more than 10-fold improvement over the benchmark and a more than 8.5-fold improvement over the SGM containing formulation Exs. 32-34. Thus, the silicone elastomer blend containing formulation examples deliver IBP significantly better than both the commercial benchmark Ibutop 5% and the SGM containing formulations.

### Examples 35-39

Formulation Ex. 35 was prepared by weighing 0.0504 g of SGM in a scintillation vial followed by addition of 0.0505 g of HCO and 9.9046 g of HMDS. The vial was closed with a lid and mixed using a vortex mixer. The HCO was not dissolved, but was dispersed in the solution. Formulation Exs. 36 and 37 were prepared using a similar procedure to that described above by changing the amount of individual components as shown below in Table 8.

Silicone elastomer based formulation Exs. 38 and 39 were prepared similar to Exs. 2 and 3A, respectively. HCO was used in formulation Exs. 38 and 39 instead of IBP in formulation Exs. 2 and 3A. The compositions of Exs. 35-39 are presented in Table 9 below.

TABLE 9
Composition of Formulation Examples 35-39.

| | Formulation examples | | | | |
|---|---|---|---|---|---|
| | 35 | 36 | 37 | 38 | 39 |
| Ingredients | % (w/w) | | | | |
| SGM | 0.5 | 1.0 | 2.0 | — | — |
| HMDS | 99.0 | 98.5 | 97.5 | — | — |
| Silicone Organic Elastomer only | — | — | — | 15.7 | |
| (from SEB1) | | | | | |
| Silicone Elastomer only (from | — | — | — | — | 19.4 |
| SEB2) | | | | | |
| Isododecane (from SEB1) | — | — | — | 44.4 | — |
| Cyclopentasiloxane (from | — | — | — | — | 55.1 |
| SEB2) | | | | | |
| PG | — | — | — | 9.6 | 6.8 |
| OLAC | — | — | — | 1.1 | 0.7 |
| IPA | — | — | — | 28.8 | 17.5 |
| HCO | 0.5 | 0.5 | 0.5 | 0.5 | 0.5 |
| Total | 100.0 | 100.0 | 100.0 | 100.0 | 100.0 |

The cumulative amount of HCO delivered across the skin, (or the total amount of HCO delivered through skin per unit area (ng/cm²) for the entire experimental period of 24 hours) by the formulations 35-39 was determined using Franz cell permeability experiment set-up at 32° C. using epidermis of human cadaver skin as described earlier. A benchmark product (Hydrocortisone 0.5% cream) was also included in the experiment. The permeability experiment was conducted using same conditions at the same time using the same skin epidermis for all the formulations.

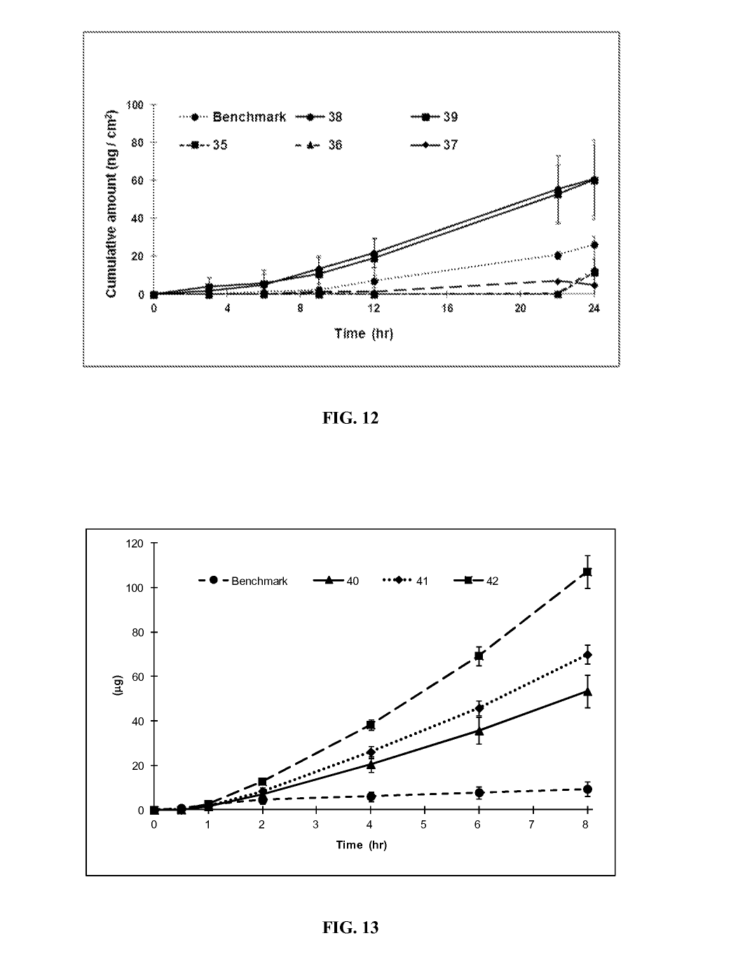

The flux profile showing the cumulative amount of the drug delivered to the membrane during the 24 hour experiment for the formulation Exs. 35-39 is provided in FIG. 12. FIG. 12 also shows the cumulative amount of the drug delivered for benchmark product (Hydrocortisone 0.5% cream), applied in the same amount, to the same amount (area) of the skin membrane. The permeability experiment was carried out at the same time using the same conditions for all the formulations and for the benchmark. 10 mg of each formulation and the benchmark was applied to the skin of Donor 12 (see Table 10 below).

As seen in FIG. 12 and Table 10, the silicone elastomer blend containing formulations 38 and 39 deliver the HCO to the membrane significantly better than the SGM containing formulation Exs. 35-37 and the benchmark Hydrocortisone 0.5% cream. After 24 hours, the benchmark delivered about 18 ng or 0.034% by weight of HCO to the membrane. SGM containing formulation Exs. 35 and 36 delivered about 7 and 8 ng, respectively, or about 0.014 and 0.016% by weight of the drug to the membrane, respectively. Formulation Ex. 37 delivered about 4 ng or about 0.0084% by weight of the drug to the membrane. SEB1 containing formulation Ex. 38 delivered about 38 ng or about 0.073% by weight of HCO to the membrane, a more than 2-fold improvement over the benchmark and a more than 4.5-fold improvement over the SGM containing formulation Exs. 35-37 (an almost 9-fold improvement over formulation Ex. 37). SEB2 containing formulation Ex. 39 delivered about 28 ng or about 0.053% by weight of HCO to the membrane, a more than 1.5-fold improvement over the benchmark and a more than 3-fold improvement over the SGM containing formulation Exs. 35-37 (and a more than 6-fold improvement over formulation Ex. 37). Thus, the silicone elastomer blend containing formulation examples deliver HCO significantly better than both the commercial benchmark Hydrocortisone 0.5% and the SGM containing formulations.

TABLE 10
Cumulative Amount and % Drug Release for Formulation Examples 22-42
and Corresponding Benchmark.

| | Cumulative | Total | | |
|---|---|---|---|---|
| | amount | Time | Drug release | Skin |
| Formulation | delivered | (hrs) | (% wt.) | epidermis |
| | (μg) | | | |
| Benchmark_DCF | 2.67 | 8 | 1.33 | Donor_9 |
| 22 | 50.06 | 8 | 25.03 | (FIGS. 8, 9) |
| 23 | 22.53 | 8 | 11.26 | |
| 24 | 7.83 | 8 | 3.91 | |
| 25 | 9.93 | 8 | 4.96 | |
| 26 | 70.48 | 8 | 35.24 | |
| 27 | 21.4 | 8 | 10.7 | |
| 28 | 13.31 | 8 | 6.65 | |
| | (ng) | | | |
| Benchmark_CLP | 2114.07 | 30 | 21.14 | Donor_10 |
| 29 | 2498.32 | 30 | 24.98 | (FIG. 10) |
| 30 | 5324.06 | 30 | 53.24 | |
| 31 | 517.67 | 30 | 5.17 | |
| | (μg) | | | |
| Benchmark_IBP | 4.13 | 24 | 1.17 | Donor_11 |
| 32 | 7.13 | 24 | 1.35 | (FIG. 11) |
| 33 | 7.26 | 24 | 1.38 | |
| 34 | 4.99 | 24 | 0.95 | |
| 2 | 47.03 | 24 | 9.9 | |
| 3A | 63.11 | 24 | 11.58 | |
| | (ng) | | | |
| Benchmark_HCO | 18.37 | 24 | 0.034 | Donor_12 |
| 35 | 7.38 | 24 | 0.014 | (FIG. 12) |
| 36 | 8.43 | 24 | 0.016 | |
| 37 | 4.42 | 24 | 0.0084 | |
| 38 | 38.46 | 24 | 0.073 | |
| 39 | 27.81 | 24 | 0.053 | |
| | (μg) | | | |
| Benchmark IBP | 9.5 | 8 | 0.94 | Donor_13 |
| 40 | 53.3 | 8 | 13.34 | (FIG. 13) |
| 41 | 69.8 | 8 | 11.64 | |
| 42 | 107.1 | 8 | 13.38 | |

Silicone elastomer based formulation Exs. 40-42 were prepared similar to formulation Ex. 2 using the same formulation composition but with different IBP concentration. IBP concentration in formulation Exs. 40, 41, and 42 was 2, 3, and 4%, respectively. The compositions of formulations Exs. 40-42 are presented in Table 11 below.

TABLE 11
Composition of formulation Exs. 40-42.

| | Formulation examples | | |
|---|---|---|---|
| | 40 | 41 | 42 |
| Ingredients | % (w/w) | | |
| Silicone Organic Elastomer only (from SEB1) | 15.5 | 15.4 | 15.2 |
| Isododecane only (from SEB1) | 43.8 | 43.3 | 42.8 |
| PG | 9.4 | 9.3 | 9.2 |
| OLAC | 1.0 | 1.0 | 1.0 |
| IPA | 28.3 | 28.1 | 27.8 |
| IBP | 2.0 | 3.0 | 4.0 |
| Total | 100.0 | 100.0 | 100.0 |

Similar to that carried out for Exs. 1, 2, and 3, the flux experiment was carried out for Exs. 40-42 and the benchmark. FIG. 13 shows the flux profile for formulation Exs. 40-42 along with that for the benchmark (Ibutop 5% gel). The flux experiment was carried out at the same time using the same conditions for all the formulations and the bench mark. About 20 mg of the formulations prepared in Exs. 40-42 was applied to the membrane of Donor 13.

As seen in FIG. 13, the cumulative amount of release of formulations prepared in Exs. 40, 41, and 42 containing 2, 3, and 4% w/w IBP, respectively, was higher than that showed by the commercial benchmark containing 5% IBP. As seen in Table 10, formulations 40, 41, and 42 resulted in a cumulative release of 53, 69, and 107 μg of IBP, respectively, which represents about 11-13% by weight of the drug after 8 hours compared to 9.5 μg cumulative release exhibited by the benchmark containing 5% IBP, which represents only about 0.94% by weight of the drug that is present in the benchmark. Thus, the cumulative release after 8 hours of the silicone organic elastomer blend containing formulation Ex. 40, that includes 2% IBP is over five times higher than that of the commercial benchmark including 5% IBP. The cumulative release after 8 hours of the silicone organic elastomer blend containing formulation Ex. 41 that includes 3% IBP is about seven times higher than that of the commercial benchmark including 5% IBP. The cumulative release after 8 hours of the silicone organic elastomer blend containing formulation Ex. 42 that includes 4% IBP is about eleven times higher than that of the commercial benchmark including 5% IBP. Thus, silicone elastomer blend containing formulations including lower concentrations of IBP than the benchmark deliver IBP significantly better than the commercial benchmark.

While the present disclosure is susceptible to various modifications and alternative forms, specific embodiments have been shown by way of example in the examples and described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

What is claimed is:

1. An anhydrous semi-solid topical drug delivery formulation comprising:
(a) a silicone-based excipient selected from a dimethicone cross polymer, a dimethicone/bis-isobutyl propylene glycol cross polymer, a polyethylene glycol-12 dimethicone/bis-isobutyl propylene glycol-20 cross polymer, or any combination thereof;
(b) at least one volatile solvent;
(c) at least one active configured to be topically delivered through a patient's skin for an intended therapeutic application;
(d) at least one enhancer; and
(e) optionally, at least one agent configured to provide occlusivity when the formulation is applied on the patient's skin.

2. (canceled)

3. The formulation of claim 1, wherein (a) is included in a carrier fluid selected from isododecane, cyclopentasiloxane, isodecylneopentanoate, and caprylyl methicone.

4. The formulation of claim 1, wherein (d) is propylene glycol, butylene glycol, dipropylene glycol, polyethylene glycol-20, oleic acid, oleyl alcohol, isopropyl myristate, dimethylisosorbide, dimethyl sulfoxide, or any combination thereof.

5. The formulation of any one of claims 1 or 4, wherein (d) includes a non-volatile excipient and a skin penetration enhancer, wherein the weight ratio of the non-volatile excipient to the penetration enhancer is optionally from about 100:1 to about 50:50.

6. The formulation of claims 1 or 3, wherein (b) is isopropyl alcohol, ethanol, ethyl acetate, hexamethyldisiloxane, polydimethylsiloxane, water, or any combination thereof.

7. The formulation of claims 1 or 3, wherein (e) is petrolatum, organic wax, silicone wax, or any combination thereof.

8. The formulation of claims 1 or 3, wherein (c) is a non-steroidal anti-inflammatory drug, a steroid, a retinoid, an azole, traditional Chinese medicines, anti-acne, antibiotics, or any combination thereof.

9. (canceled)

10. (canceled)

11. The formulation of claim 1, wherein the formulation is anhydrous.

12. The formulation of claims 1 or 8, being free of preservatives.

13. The formulation of claim 1, being configured to deliver a therapeutically active amount of the at least one active to the patient's skin for an extended period of time.

14. The formulation of claim 1, being configured to deliver a therapeutically active amount of the at least one active to the patient's skin for more than 4 hours or for more than 8 hours.

15. A method for increasing the penetration of a pharmaceutical active ingredient through the skin of a mammal, comprising topically administering to the skin a chemically and physically stable formulation of claim 1.

16. The formulation of claim 1, wherein
(b) is isopropyl alcohol, ethanol, ethyl acetate, hexamethyldisiloxane, polydimethylsiloxane, water, or any combination thereof;
(c) is a non-steroidal anti-inflammatory drug, a steroid, a retinoid, an azole, traditional Chinese medicines, anti-acne, antibiotics, or any combination thereof;
(d) is propylene glycol, butylene glycol, dipropylene glycol, polyethylene glycol-20, oleic acid, oleyl alcohol, isopropyl myristate, dimethylisosorbide, dimethyl sulfoxide, or any combination thereof; and
(e) is petrolatum, organic wax, silicone wax, or any combination thereof.
